SEQUENCE LISTING (12 sequences)

SEQ ID NO: 1 - protein, 38 aa, synthetic construct
  REGION 1..38: note = Description of Artificial Sequence: Synthetic polypeptide
  VARIANT 2: note = P, A, or V
  VARIANT 4..31: note = X can be any amino acid
  SITE 4..31: note = This region may encompass 23-28 residues
  VARIANT 34..37: note = X can be any amino acid
  SITE 34..37: note = This region may encompass 2-4 residues
HXEXXXXXXXXXXXXXXXXXXXXXXXXXXXXPCXXXXC

SEQ ID NO: 2 - protein, 87 aa, synthetic construct
  REGION 1..87: note = Description of Artificial Sequence: Synthetic polypeptide
  VARIANT 2: note = X can be any amino acid
  VARIANT 4..27: note = X can be any amino acid
  VARIANT 30: note = S or T
  VARIANT 33..36: note = X can be any amino acid
  SITE 33..36: note = This region may encompass 2-4 residues
  VARIANT 45..52: note = X can be any amino acid
  VARIANT 54..58: note = X can be any amino acid
  VARIANT 60: note = L or I
  VARIANT 62..72: note = X can be any amino acid
  SITE 62..72: note = This region may encompass 8-11 residues
  VARIANT 74..75: note = X can be any amino acid
  VARIANT 77..86: note = X can be any amino acid
  VARIANT 38..43: note = X can be any amino acid
HXEXXXXXXXXXXXXXXXXXXXXXXXXSWXPCXXXXCXXXXXXFXXXXXXXXLXXXXXRXYXXXXXXXXXXXLXXLXXXXXXXXXXM

SEQ ID NO: 3 - protein, 199 aa, synthetic construct
  REGION 1..199: note = Description of Artificial Sequence: Synthetic polypeptide
  VARIANT 1..26: note = X can be any amino acid
  SITE 1..26: note = This region may encompass 16-26 residues
  VARIANT 29..30: note = X can be any amino acid
  VARIANT 32: note = X can be any amino acid
  VARIANT 36: note = X can be any amino acid
  VARIANT 38..52: note = X can be any amino acid
  VARIANT 54..56: note = X can be any amino acid
  VARIANT 58..69: note = X can be any amino acid
  VARIANT 73..74: note = X can be any amino acid
  VARIANT 76..89: note = X can be any amino acid
  VARIANT 91..92: note = X can be any amino acid
  VARIANT 95..96: note = X can be any amino acid
  VARIANT 102..105: note = X can be any amino acid
  SITE 102..105: note = This region may encompass 2-4 residues
  VARIANT 108..112: note = X can be any amino acid
  VARIANT 115..121: note = X can be any amino acid
  VARIANT 123: note = X can be any amino acid
  VARIANT 125..127: note = X can be any amino acid
  VARIANT 129: note = L or I
  VARIANT 131..138: note = X can be any amino acid
  VARIANT 141..142: note = X can be any amino acid
  VARIANT 144..146: note = X can be any amino acid
  VARIANT 148..152: note = X can be any amino acid
  VARIANT 154..157: note = X can be any amino acid
  VARIANT 159..160: note = X can be any amino acid
  VARIANT 163..164: note = X can be any amino acid
  VARIANT 167..172: note = X can be any amino acid
  VARIANT 174: note = X can be any amino acid
  VARIANT 177..189: note = X can be any amino acid
  VARIANT 191..192: note = X can be any amino acid
  VARIANT 194..199: note = X can be any amino acid
  SITE 194..199: note = This region may encompass 2-6 residues
XXXXXXXXXXXXXXXXXXXXXXXXXXGRXXTXLCYXVXXXXXXXXXXXXXXXGXXXNXXXXXXXXXXXXHAEXXFXXXXXXXXXXXXXXYXXTWXXSWSPCXXXXCAXXXXXFLXXXXXXXLXIXXXRXYXXXXXXXXGLXXLXXXGXXXXXMXXXXFXXCWXXFVXXXXXXFXPWXXXXXXXXXXXXXLXXIXXXXXX

SEQ ID NO: 4 - protein, 199 aa, synthetic construct
  REGION 1..199: note = Description of Artificial Sequence: Synthetic polypeptide
  VARIANT 1..26: note = X can be any amino acid
  VARIANT 29..30: note = X can be any amino acid
  VARIANT 32: note = X can be any amino acid
  VARIANT 36: note = X can be any amino acid
  VARIANT 38..52: note = X can be any amino acid
  VARIANT 54..69: note = X can be any amino acid
  VARIANT 73..74: note = X can be any amino acid
  VARIANT 76..89: note = X can be any amino acid
  VARIANT 91..92: note = X can be any amino acid
  VARIANT 95..96: note = X can be any amino acid
  VARIANT 102..105: note = X can be any amino acid
  VARIANT 108..112: note = X can be any amino acid
  VARIANT 115..121: note = X can be any amino acid
  VARIANT 123: note = X can be any amino acid
  VARIANT 126..127: note = X can be any amino acid
  VARIANT 129: note = L or I
  VARIANT 131..138: note = X can be any amino acid
  VARIANT 141..142: note = X can be any amino acid
  VARIANT 144..146: note = X can be any amino acid
  VARIANT 148..152: note = X can be any amino acid
  VARIANT 154..157: note = X can be any amino acid
  VARIANT 159..160: note = X can be any amino acid
  VARIANT 163..164: note = X can be any amino acid
  VARIANT 167..172: note = X can be any amino acid
  VARIANT 174: note = X can be any amino acid
  VARIANT 177..189: note = X can be any amino acid
  VARIANT 194..199: note = X can be any amino acid
  VARIANT 191..192: note = X can be any amino acid
XXXXXXXXXXXXXXXXXXXXXXXXXXGRXXTXLCYXVXXXXXXXXXXXXXXXGXXXXXXXXXXXXXXXXHAEXXFXXXXXXXXXXXXXXYXXTWXXSWSPCXXXXCAXXXXXFLXXXXXXXLXIFXXRXYXXXXXXXXGLXXLXXXGXXXXXMXXXXFXXCWXXFVXXXXXXFXPWXXXXXXXXXXXXXLXXIXXXXXX

SEQ ID NO: 5 - protein, 199 aa, synthetic construct
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIADYDPLYKEALQMLRDAGAQVSIMTYDEFKHCWDTFVDHQGCPFQPWDGLDEHSQALSGRLRAILQNQGN

SEQ ID NO: 6 - protein, 199 aa, synthetic construct
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIADHDPLYKEALQMLRDAGAQVSIMTYDEFKHCWDTFVDHQGCPFQPWDGLDEHSQALSGRLRAILQNQGN

SEQ ID NO: 7 - protein, 199 aa, synthetic construct
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARILDYDPLYKEALQMLRDAGAQVSIMTYDEFKHCWDTFVDHQGCPFQPWDGLDEHSQALSGRLRAILQNQGN

SEQ ID NO: 8 - protein, 199 aa, synthetic construct
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIWDYDPLYKEALQMLRDAGAQVSIMTYDEFKHCWDTFVDHQGCPFQPWDGLDEHSQALSGRLRAILQNQGN

SEQ ID NO: 9 - protein, 199 aa, synthetic construct
  VARIANT 130: note = X can be A, G, F, H, Q, M, N, K, V, D, E, S, C, P, or T.
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIXDYDPLYKEALQMLRDAGAQVSIMTYDEFKHCWDTFVDHQGCPFQPWDGLDEHSQALSGRLRAILQNQGN

SEQ ID NO: 10 - protein, 199 aa, synthetic construct
  VARIANT 128: note = B can be L or I.
  VARIANT 129: note = Z can be A, G, F, H, Q, M, N, K, V, D, E, S, C, P, or T.
XXXXXXXXXXXXXXXXXXXXXXXXXXGRXXTXLCYXVXXXXXXXXXXXXXXXGXXXXXXXXXXXXXXXXHAEXXFXXXXXXXXXXXXXXYXXTWXXSWSPCXXXXCAXXXXXFLXXXXXXXLXIFXXRBZXXXXXXXXGLXXLXXXGXXXXXMXXXXFXXCWXXFVXXXXXXFXPWXXXXXXXXXXXXXLXXIXXXXXX

SEQ ID NO: 11 - protein, 199 aa, synthetic construct
  VARIANT 130: note = X can be A, L, or W, preferably A.
  VARIANT 132: note = Z can be R, H, L, or Q, preferably H.
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIXDZDPLYKEALQMLRDAGAQVSIMTYDEFKHCWDTFVDHQGCPFQPWDGLDEHSQALSGRLRAILQNQGN

SEQ ID NO: 12 - protein, 199 aa, synthetic construct
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

(19) United States
(12) Patent Application Publication
(10) Pub. No.: US 2025/0327067 A1
(43) Pub. Date: Oct. 23, 2025

(54) REDUCING URACILS BY POLYMERASE

(71) Applicant: ILLUMINA, INC., San Diego, CA (US)

(72) Inventors: Kayla Busby, San Diego, CA (US); Allison Kathleen Yunghans, San Diego, CA (US); Angelica Marie Barr Schalembier, San Diego, CA (US); Stephen Gross, San Diego, CA (US)

(73) Assignee: ILLUMINA, INC., San Diego, CA (US)

(21) Appl. No.: 18/850,719

(22) PCT Filed: Dec. 15, 2023
(86) PCT No.: PCT/US2023/084217

§ 371 Date: Sep. 25, 2024

Related U.S. Application Data
(60) Provisional application No. 63/437,413, filed on Jan. 6, 2023.

(51) Int. Cl. C12N 15/10

(52) U.S. Cl. CPC C12N 15/1093

(57) ABSTRACT

Described herein are methods of removing false positive uracils due to the deamination of unmethylated cytosines in assays using engineered cytosine deaminases to deaminate methylated cytosines, the methods utilizing enzymes that discriminate against uracil residues, such as for example, uracil-intolerant polymerases, uracil DNA glycosylase (UDG), and/or USER™ (Uracil-Specific Excision Reagent) enzyme, to remove false positive uracil residues from cytidine deaminase mediated methylation sequencing assays.

### CONTINUING APPLICATION DATA

This application claims the benefit of U.S. Provisional Application Ser. No. 63/437,413, filed Jan. 6, 2023, which is incorporated by reference herein.

### SEQUENCE LISTING

This application contains a Sequence Listing electronically submitted to the United States Patent and Trademark Office as an XML file entitled “531_002461WO01_ST26.xml” having a size of 46 kilobytes and created on Dec. 8, 2023. The information contained in the Sequence Listing is incorporated by reference herein.

### FIELD

Embodiments of the present disclosure relate to the prevention of false positive detection of 5-methylcytosine (5mC) and/or 5-hydroxymethylcytosine (5hmC) due to the deamination of unmethylated cytosines in assays using cytosine deaminases to selectively deaminate methylated cytosines. In particular, embodiments of the methods, compositions, and kits provided herein utilize an uracil-intolerant polymerase and/or treatment with Uracil DNA Glycosylase (UDG) in order to reduce the likelihood that such false positive conversions are detected in the final sequenced library.

### BACKGROUND

Modified DNA cytosines, including 5-methylcytosine (5mC), are a well-studied epigenetic modification that play fundamental roles in human development and disease. Its genome-wide distribution differs between tissue types, and between healthy and diseased states. In recent years, 5mC has also gained prominence as a tool for clinical diagnostics. For example, its distribution in cell-free DNA (cfDNA) obtained from a liquid biopsy can be used for the tissue-specific prediction of early-stage cancer. As a result, there has been an intense focus on developing methods for mapping 5mC at single base resolution, with minimal loss of sample DNA quantity, quality, and complexity.

5mC bases treated with a cytosine deaminase result in thymine bases, providing a signal for assessing sequence-specific methylation state of cytosines when sequenced. APOBEC3A is a cytidine deaminase that recognizes single-stranded DNA and catalyzes the deamination of cytosine (C) to uracil (U), 5-methylcytosine (5mC) to thymine (T), and 5-hydroxymethylcytosine to 5-hydroxymethyluracil. Protein engineering of APOBEC3A has resulted in mutant APOBEC proteins with selectivity towards deamination of 5mC with reduced activity towards deamination of C, however residual activity for deamination of C remains. This undesirable deamination of unmethylated cytosines results in the false positive detection of 5mC (and 5hmC) with uracil bases being read as thymine bases in the assay.

### SUMMARY OF THE INVENTION

In one aspect, this disclosure describes a method of preventing false positive detection of 5-methylcytosine (5mC) and/or 5-hydroxymethylcytosine (5hmC) due to deamination of unmethylated cytosines in an assay using a cytosine deaminase to selectively deaminate methylated cytosines, the method including: providing a sample including DNA library fragments in which a cytosine deaminase has deaminated methylated cytosines, wherein the DNA library fragments include 5′ end and 3′ end library adapters; and subjecting the sample to at least one round of second strand synthesis by contacting the sample including DNA library fragments with an uracil-intolerant polymerase, dNTPs, and primers complementary to the 5′ end and 3′ end library adapters under conditions to provide for second strand synthesis resulting in double stranded DNA uracil-free library fragments. In some aspects, the method further includes subjecting the sample of double stranded DNA uracil-free library fragments to polymerase chain reaction (PCR) amplification with either a uracil-tolerant polymerase or a uracil-intolerant polymerase. In some aspects, the uracil-intolerant polymerase includes KAPA HiFi, Ultra II Q5, or Phusion HiFi.

In one aspect, this disclosure describes a method of preventing false positive detection of 5-methylcytosine (5mC) and/or 5-hydroxymethylcytosine (5hmC) due to deamination of unmethylated cytosines in an assay using a cytosine deaminase to selectively deaminate methylated cytosines, the method including: providing a sample including library DNA fragments in which a cytosine deaminase has deaminated methylated cytosines, wherein the DNA library fragments include 5′ end and 3′ end library adapters; and contacting the sample including DNA library fragments with an uracil DNA glycosylase (UDG) and an endonuclease resulting in DNA library fragments cleaved at uracil residues; and subjecting the sample including DNA library fragments cleaved at uracil residues to polymerase chain reaction (PCR) amplification by contacting the sample including DNA library fragments cleaved at uracil residues with a polymerase, dNTPs, and primers complementary to the 5′ end and 3′ end library adapters under conditions to provide for second strand synthesis resulting in double stranded DNA uracil-free library fragments. In some aspects, the polymerase includes an uracil-intolerant polymerase. In some aspects, the uracil-intolerant polymerase includes KAPA HiFi, Ultra II Q5, or Phusion HiFi.

In one aspect, this disclosure describes a method of preventing false positive detection of 5-methylcytosine (5mC) and/or 5-hydroxymethylcytosine (5hmC) due to deamination of unmethylated cytosines in an assay using a cytosine deaminase to selectively deaminate methylated cytosines, the method including: providing a sample including original DNA library fragments in which a cytosine deaminase has deaminated methylated cytosines, wherein the original DNA library fragments includes 5′ end and 3′ end library adapters; subjecting the sample to second strand synthesis by contacting the sample including original DNA library fragments with an uracil-intolerant polymerase, dNTPs, and primers complementary to the 5′ end and 3′ end library adapters under conditions to provide for second strand synthesis resulting in DNA library fragments without uracil residues in the synthesized second strand; contacting the sample including DNA library fragments without uracil residues in the synthesized second strand with an endonuclease to digest the original DNA library fragments resulting in single stranded synthesized second strands; and subjecting the sample including single stranded synthesized second strands to polymerase chain reaction (PCR) amplification by contacting the sample including single stranded synthesized second strands with a polymerase, dNTPs, and primers complementary to the 5′ end and 3′ end library adapters under conditions to provide for second strand synthesis resulting in double stranded DNA uracil-free library fragments. In some aspects, the uracil-intolerant polymerase includes KAPA HiFi, Ultra II Q5, or Phusion HiFi. In some aspect, subjecting the sample including single stranded synthesized second strands to PCR amplification includes contacting the sample including single stranded synthesized second strands with a uracil tolerant polymerase.

In one aspect, this disclosure describes a method of removing DNA fragments including uracil residues from a sample, the method including: providing a sample including DNA fragments, wherein the DNA fragments include 5′ end and 3′ end library adapters; and subjecting the sample to polymerase chain reaction (PCR) amplification by contacting the sample including DNA fragments with an uracil-intolerant polymerase, dNTPs, and primers complementary to the 5′ end and 3′ end library adapters under conditions to provide for second strand synthesis resulting in double stranded DNA uracil-free fragments. In some aspects, the method further includes subjecting the sample of double stranded DNA uracil-free library fragments to polymerase chain reaction (PCR) amplification with either a uracil-tolerant polymerase or a uracil-intolerant polymerase. In some aspects, the uracil-intolerant polymerase includes KAPA HiFi, Ultra II Q5, or Phusion HiFi.

In one aspect, this disclosure describes a method of removing DNA fragments including uracil residues from a sample, the method including: providing a sample including DNA fragments, wherein the DNA fragments includes 5′ end and 3′ end library adapters; and contacting the sample including DNA fragments with an uracil DNA glycosylase (UDG) and an endonuclease resulting in DNA fragments cleaved at uracil residues; and subjecting the sample including DNA fragments cleaved at uracil residues to polymerase chain reaction (PCR) amplification by contacting the sample including DNA fragments cleaved at uracil residues with a polymerase, dNTPs, and primers complementary to the 5′ end and 3′ end library adapters under conditions to provide for second strand synthesis resulting in double stranded DNA uracil-free fragments. In some aspects, the polymerase includes an uracil intolerant polymerase. In some aspects, the polymerase includes an uracil-intolerant polymerase. In some aspects, the uracil-intolerant polymerase includes KAPA HiFi, Ultra II Q5, or Phusion HiFi.

In one aspect, this disclosure describes a method of removing DNA fragments including uracil residues from a sample, the method including: providing a sample including original DNA fragments, wherein the original DNA fragments include 5′ end and 3′ end library adapters; subjecting the sample to second strand synthesis by contacting the sample including original DNA fragments with an uracil-intolerant polymerase, dNTPs, and primers complementary to the 5′ end and 3′ end library adapters under conditions to provide for second strand synthesis resulting in DNA fragments without uracil residues in the synthesized second strand; contacting the sample including DNA fragments without uracil residues in the synthesized second strand with an endonuclease to digest the original DNA fragments resulting in single stranded synthesized second strands; and subjecting the sample including single stranded synthesized second strands to polymerase chain reaction (PCR) amplification by contacting the sample including single stranded synthesized second strands with a polymerase, dNTPs, and primers complementary to the 5′ end and 3′ end library adapters under conditions to provide for second strand synthesis resulting in double stranded DNA uracil-free fragments. In some aspects, the uracil-intolerant polymerase includes KAPA HiFi, Ultra II Q5, or Phusion HiFi. In some aspect, subjecting the sample including single stranded synthesized second strands to PCR amplification includes contacting the sample including single stranded synthesized second strands with a uracil tolerant polymerase.

In some aspects of the methods disclosed herein, the DNA library fragments include single stranded DNA library fragments. In some aspects, the DNA library fragments include double stranded DNA library fragments.

In some aspects of the methods disclosed herein, the cytosine deaminase includes an altered cytosine deaminase.

In some aspects, the altered cytosine deaminase is a member of the AID subfamily, the APOBEC1 subfamily, the APOBEC2 subfamily, the APOBEC3A subfamily, the APOBEC3B subfamily, the APOBEC3C subfamily, the APOBEC3D subfamily, the APOBEC3F subfamily, the APOBEC3G subfamily, the APOBEC3G subfamily, the APOBEC3H subfamily, or the APOBEC4 subfamily, or an alteration thereof. In some aspects, the altered cytosine deaminase comprises an altered APOBEC3A.

In some aspects, the altered cytidine deaminase comprises an amino acid substitution mutation at a position functionally equivalent to (Tyr/Phe) 130 in a wild-type APOBEC3A protein and/or an amino acid substitution mutation at a position functionally equivalent to Tyr132 in a wild-type APOBEC3A protein. In some aspects, the altered cytidine deaminase comprises amino acid substitution mutations at positions functionally equivalent to (Tyr/Phe) 130 and Tyr132 in a wild-type APOBEC3A protein.

In some aspects, the substitution mutation at the position functionally equivalent to Tyr130 comprises a mutation to alanine, glycine, phenylalanine, histidine, glutamine, methionine, asparagine, lysine, valine, aspartic acid, glutamic acid, serine, cysteine, proline, arginine, or threonine.

In some aspects, the substitution mutation at the position functionally equivalent to Tyr130 comprises a mutation to Ala, Val, or Trp.

In some aspects, the substitution mutation at the position functionally equivalent to Tyr132 comprises a mutation to His, Arg, Gln, or Lys.

In some aspects, the altered cytidine deaminase comprises an amino acid substitution mutation at a position functionally equivalent to (Tyr/Phe) 130 in a wild-type APOBEC3A protein, wherein the substitution mutation is (Tyr/Phe) 130Trp.

In some aspects, the (Tyr/Phe) 130 is Tyr130, and the wild-type APOBEC3A protein is SEQ ID NO: 12.

In some aspects, the altered cytidine deaminase converts 5-methyl cytosine (5mC) to thymidine (T) by deamination at a greater rate than conversion of cytosine (C) to uracil (U) by deamination. In some aspects, the rate is at least 100-fold greater.

In some aspects, the altered cytidine deaminase converts cytosine (C) to uracil (U) by deamination and 5-methyl cytosine (5mC) to thymidine (T) by deamination at a greater rate than conversion of 5-hydroxymethyl cytosine (5hmC) to 5-hydroxymethyl uracil (5hmU) by deamination. In some aspects, conversion of 5hmC to 5hmU by deamination is undetectable.

In some aspects, the altered cytidine deaminase comprises a ZDD motif H-[P/A/V]-E-X[23-28]-P—C-X[2-4]-C(SEQ ID NO: 1).

In some aspects, the altered cytidine deaminase is a member of the APOBEC3A subfamily and comprises a ZDD motif HXEX₂₄SW(S/T)PCX[2-4]CX6FX8LX5R(L/I)YX[8-11]LX2LX[10]M (SEQ ID NO: 2), wherein the amino acid substitution mutation at the position functionally equivalent to (Tyr/Phe) 130 of the wild-type APOBEC3A protein is the Tyr (Y) amino acid of the ZDD motif.

In some aspects, the altered cytidine deaminase is a member of the APOBEC3A subfamily and comprises X[16-26]-GRXXTXLCYXV-X15-GXXXN-X12-HAEXXF-X14-YXXTWXXSWSPC-X[2-4]-CA-X5-FL-X7-LXIXXXR(L/I)Y-X8-GLXXLXXXG-X5-M-X4-FXXCWXXFV-X6-FXPW-X13-LXXI-X[2-6] (SEQ ID NO: 3).

In some aspects, the altered cytidine deaminase is a member of the APOBEC3A family and comprises SEQ ID NO: 5, SEQ ID NO: 6, SEQ ID NO: 7, SEQ ID NO: 8, SEQ ID NO: 9, SEQ ID NO: 10, or SEQ ID NO: 11.

In some aspects of the methods disclosed herein, the DNA library fragments are about 100 bp to about 300 bp in length.

In some aspects of the methods disclosed herein, the method further includes sequencing the double stranded DNA uracil-free library fragments.

In some aspects of the methods disclosed herein, the method further includes processing the double stranded DNA uracil-free library fragments to produce a sequencing library. In some aspects, the method also further includes sequencing the sequencing library.

In one aspect, this disclosure describes a kit including a cytosine deaminase and an uracil intolerant polymerase. In some aspects, the uracil-intolerant polymerase includes KAPA HiFi, Ultra II Q5, or Phusion HiFi. In some aspects, the cytosine deaminase includes an altered cytosine deaminase. In some aspects, the altered cytosine deaminase is a member of the AID subfamily, the APOBEC1 subfamily, the APOBEC2 subfamily, the APOBEC3A subfamily, the APOBEC3B subfamily, the APOBEC3C subfamily, the APOBEC3D subfamily, the APOBEC3F subfamily, the APOBEC3G subfamily, the APOBEC3G subfamily, the APOBEC3H subfamily, or the APOBEC4 subfamily, or an alteration thereof. In some aspects, the altered cytosine deaminase comprises an altered APOBEC3A. In some aspects, the altered cytidine deaminase comprises an amino acid substitution mutation at a position functionally equivalent to (Tyr/Phe) 130 in a wild-type APOBEC3A protein and/or an amino acid substitution mutation at a position functionally equivalent to Tyr132 in a wild-type APOBEC3A protein. In some aspects, the altered cytidine deaminase comprises amino acid substitution mutations at positions functionally equivalent to (Tyr/Phe) 130 and Tyr132 in a wild-type APOBEC3A protein. In some aspects, the substitution mutation at the position functionally equivalent to Tyr130 comprises a mutation to alanine, glycine, phenylalanine, histidine, glutamine, methionine, asparagine, lysine, valine, aspartic acid, glutamic acid, serine, cysteine, proline, arginine, or threonine. In some aspects, the substitution mutation at the position functionally equivalent to Tyr130 comprises a mutation to Ala, Val, or Trp. In some aspects, the substitution mutation at the position functionally equivalent to Tyr132 comprises a mutation to His, Arg, Gln, or Lys. In some aspects, the altered cytidine deaminase comprises an amino acid substitution mutation at a position functionally equivalent to (Tyr/Phe) 130 in a wild-type APOBEC3A protein, wherein the substitution mutation is (Tyr/Phe) 130Trp. In some aspects, the (Tyr/Phe) 130 is Tyr130, and the wild-type APOBEC3A protein is SEQ ID NO: 12. In some aspects, the altered cytidine deaminase converts 5-methyl cytosine (5mC) to thymidine (T) by deamination at a greater rate than conversion of cytosine (C) to uracil (U) by deamination. In some aspects, the rate is at least 100-fold greater. In some aspects, the altered cytidine deaminase converts cytosine (C) to uracil (U) by deamination and 5-methyl cytosine (5mC) to thymidine (T) by deamination at a greater rate than conversion of 5-hydroxymethyl cytosine (5hmC) to 5-hydroxymethyl uracil (5hmU) by deamination. In some aspects, conversion of 5hmC to 5hmU by deamination is undetectable. In some aspects, the altered cytidine deaminase comprises a ZDD motif H-[P/A/V]-E-X[23-28]-P—C-X[2-4]-C(SEQ ID NO: 1). In some aspects, the altered cytidine deaminase is a member of the APOBEC3A subfamily and comprises a ZDD motif HXEX₂₄SW(S/T)PCX[2-4]CX6FX8LX5R(L/I)YX[8-11]LX2LX[10]M (SEQ ID NO: 2), wherein the amino acid substitution mutation at the position functionally equivalent to (Tyr/Phe) 130 of the wild-type APOBEC3A protein is the Tyr (Y) amino acid of the ZDD motif. In some aspects, the altered cytidine deaminase is a member of the APOBEC3A subfamily and comprises X[16-26]-GRXXTXLCYXV-X15-GXXXN-X12-HAEXXF-X14-YXXTWXXSWSPC-X[2-4]-CA-X5-FL-X7-LXIXXXR(L/I)Y-X8-GLXXLXXXG-X5-M-X4-FXXCWXXFV-X6-FXPW-X13-LXXI-X[2-6] (SEQ ID NO: 3). In some aspects, the altered cytidine deaminase is a member of the APOBEC3A family and comprises SEQ ID NO: 5, SEQ ID NO: 6, SEQ ID NO: 7, SEQ ID NO: 8, SEQ ID NO: 9, SEQ ID NO: 10, or SEQ ID NO: 11.

In one aspect, this disclosure describes a kit including a cytosine deaminase, an uracil DNA glycosylase (UDG), and an AP endonuclease. In some aspects, the uracil-intolerant polymerase includes KAPA HiFi, Ultra II Q5, or Phusion HiFi. In some aspects, the AP endonuclease includes Endonuclease IV and/or AP Endonuclease I. In some aspects, the cytosine deaminase includes an altered cytosine deaminase. In some aspects, the altered cytosine deaminase is a member of the AID subfamily, the APOBEC1 subfamily, the APOBEC2 subfamily, the APOBEC3A subfamily, the APOBEC3B subfamily, the APOBEC3C subfamily, the APOBEC3D subfamily, the APOBEC3F subfamily, the APOBEC3G subfamily, the APOBEC3G subfamily, the APOBEC3H subfamily, or the APOBEC4 subfamily, or an alteration thereof. In some aspects, the altered cytosine deaminase comprises an altered APOBEC3A. In some aspects, the altered cytidine deaminase comprises an amino acid substitution mutation at a position functionally equivalent to (Tyr/Phe) 130 in a wild-type APOBEC3A protein and/or an amino acid substitution mutation at a position functionally equivalent to Tyr132 in a wild-type APOBEC3A protein. In some aspects, the altered cytidine deaminase comprises amino acid substitution mutations at positions functionally equivalent to (Tyr/Phe) 130 and Tyr132 in a wild-type APOBEC3A protein. In some aspects, the substitution mutation at the position functionally equivalent to Tyr130 comprises a mutation to alanine, glycine, phenylalanine, histidine, glutamine, methionine, asparagine, lysine, valine, aspartic acid, glutamic acid, serine, cysteine, proline, arginine, or threonine. In some aspects, the substitution mutation at the position functionally equivalent to Tyr130 comprises a mutation to Ala, Val, or Trp. In some aspects, the substitution mutation at the position functionally equivalent to Tyr132 comprises a mutation to His, Arg, Gln, or Lys. In some aspects, the altered cytidine deaminase comprises an amino acid substitution mutation at a position functionally equivalent to (Tyr/Phe) 130 in a wild-type APOBEC3A protein, wherein the substitution mutation is (Tyr/Phe) 130Trp. In some aspects, the (Tyr/Phe) 130 is Tyr130, and the wild-type APOBEC3A protein is SEQ ID NO: 12. In some aspects, the altered cytidine deaminase converts 5-methyl cytosine (5mC) to thymidine (T) by deamination at a greater rate than conversion of cytosine (C) to uracil (U) by deamination. In some aspects, the rate is at least 100-fold greater. In some aspects, the altered cytidine deaminase converts cytosine (C) to uracil (U) by deamination and 5-methyl cytosine (5mC) to thymidine (T) by deamination at a greater rate than conversion of 5-hydroxymethyl cytosine (5hmC) to 5-hydroxymethyl uracil (5hmU) by deamination. In some aspects, conversion of 5hmC to 5hmU by deamination is undetectable. In some aspects, the altered cytidine deaminase comprises a ZDD motif H-[P/A/V]-E-X[23-28]-P—C-X[2-4]-C(SEQ ID NO: 1). In some aspects, the altered cytidine deaminase is a member of the APOBEC3A subfamily and comprises a ZDD motif HXEX₂₄SW(S/T)PCX[2-4]CX6 FX8LX5R(L/I)YX[8-11]LX2LX[10]M (SEQ ID NO: 2), wherein the amino acid substitution mutation at the position functionally equivalent to (Tyr/Phe) 130 of the wild-type APOBEC3A protein is the Tyr (Y) amino acid of the ZDD motif. In some aspects, the altered cytidine deaminase is a member of the APOBEC3A subfamily and comprises X[16-26]-GRXXTXLCYXV-X15-GXXXN-X12-HAEXXF-X14-YXXTWXXSWSPC-X[2-4]-CA-X5-FL-X7-LXIXXXR(L/I)Y-X8-GLXXLXXXG-X5-M-X4-FXXCWXXFV-X6-FXPW-X13-LXXI-X[2-6] (SEQ ID NO: 3). In some aspects, the altered cytidine deaminase is a member of the APOBEC3A family and comprises SEQ ID NO: 5, SEQ ID NO: 6, SEQ ID NO: 7, SEQ ID NO: 8, SEQ ID NO: 9, SEQ ID NO: 10, or SEQ ID NO: 11.

Terms used herein will be understood to take on their ordinary meaning in the relevant art unless specified otherwise. Several terms used herein and their meanings are set forth below.

As used herein, the term “nucleic acid” is intended to be consistent with its use in the art and includes naturally occurring nucleic acids or functional analogs thereof. Particularly useful functional analogs are capable of hybridizing to a nucleic acid in a sequence specific fashion or capable of being used as a template for replication of a particular nucleotide sequence. Naturally occurring nucleic acids generally have a backbone containing phosphodiester bonds. An analog structure can have an alternate backbone linkage including any of a variety of those known in the art. Naturally occurring nucleic acids generally have a deoxyribose sugar (for example, found in deoxyribonucleic acid (DNA)) or a ribose sugar (for example, found in ribonucleic acid (RNA)). A nucleic acid can contain any of a variety of analogs of these sugar moieties that are known in the art. A nucleic acid can include native or non-native bases. In this regard, a native deoxyribonucleic acid can have one or more bases selected from the group consisting of adenine, thymine, cytosine, or guanine and a ribonucleic acid can have one or more bases selected from the group consisting of uracil, adenine, cytosine, or guanine. Useful non-native bases that can be included in a nucleic acid are known in the art. The term “template” and “target,” when used in reference to a nucleic acid, is intended as a semantic identifier for the nucleic acid in the context of a method or composition set forth herein and does not necessarily limit the structure or function of the nucleic acid beyond what is otherwise explicitly indicated.

As used herein, the term “target nucleic acid,” is intended as a semantic identifier for the nucleic acid in the context of a method or composition or kit set forth herein and does not necessarily limit the structure or function of the nucleic acid beyond what is otherwise explicitly indicated. Reference to a nucleic acid such as a target nucleic acid includes both single-stranded and double-stranded nucleic acids, and both DNA and RNA, unless indicated otherwise.

The terms “polynucleotide” and “oligonucleotide” are used interchangeably herein to refer to a polymeric form of nucleotides of any length, and may include ribonucleotides, deoxyribonucleotides, analogs thereof, or mixtures thereof. The terms should be understood to include, as equivalents, analogs of either DNA, RNA, cDNA, or antibody-oligo conjugates made from nucleotide analogs and to be applicable to single stranded (such as sense or antisense) and double stranded polynucleotides. The term as used herein also encompasses cDNA, that is complementary or copy DNA produced from an RNA template, for example by the action of reverse transcriptase.

As used herein, the term “primer” and its derivatives refer generally to any polynucleotide that can hybridize to a target sequence of interest. Typically, the primer functions as a substrate onto which nucleotides can be polymerized by a polymerase; in some embodiments, however, the primer can become incorporated into the synthesized nucleic acid strand and provide a site to which another primer can hybridize to prime synthesis of a new strand that is complementary to the synthesized nucleic acid molecule. The primer can include any combination of nucleotides or analogs thereof. In some embodiments, the primer is a single-stranded oligonucleotide or polynucleotide. The terms “polynucleotide” and “oligonucleotide” are used interchangeably herein to refer to a polymeric form of nucleotides of any length, and may comprise ribonucleotides, deoxyribonucleotides, analogs thereof, or mixtures thereof. The terms should be understood to include, as equivalents, analogs of either DNA or RNA made from nucleotide analogs and to be applicable to single stranded (such as sense or antisense) and double-stranded polynucleotides. The term as used herein also encompasses cDNA, that is complementary or copy DNA produced from an RNA template, for example by the action of reverse transcriptase. This term refers only to the primary structure of the molecule. Thus, the term includes triple-, double- and single-stranded deoxyribonucleic acid (“DNA”), as well as triple-, double- and single-stranded ribonucleic acid (“RNA”).

The term “flowcell” as used herein refers to a chamber comprising a solid surface across which one or more fluid reagents can be flowed. Examples of flowcells and related fluidic systems and detection platforms that can be readily used in the methods of the present disclosure are described, for example, in Bentley et al., 2008, Nature 456:53-59, WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082. Example flow cells and substrates for manufacture of flow cells that may be used in methods and compositions as set forth herein include, but are not limited to, those commercially available from Illumina, Inc. (San Diego, CA).

As used herein, the term “amplicon,” when used in reference to a nucleic acid, means the product of copying the nucleic acid, wherein the product has a nucleotide sequence that is the same as or complementary to at least a portion of the nucleotide sequence of the nucleic acid. An amplicon can be produced by any of a variety of amplification methods that use the nucleic acid, or an amplicon thereof, as a template including, for example, PCR, rolling circle amplification (RCA), ligation extension, or ligation chain reaction. An amplicon can be a nucleic acid molecule having a single copy of a particular nucleotide sequence (for example, a PCR product) or multiple copies of the nucleotide sequence (for example, a concatameric product of RCA). A first amplicon of a target nucleic acid is typically a complimentary copy. Subsequent amplicons are copies that are created, after generation of the first amplicon, from the target nucleic acid or from the first amplicon. A subsequent amplicon can have a sequence that is substantially complementary to the target nucleic acid or substantially identical to the target nucleic acid.

As defined herein “multiplex amplification” refers to selective and non-random amplification of two or more target sequences within a sample using at least one target-specific primer. In some embodiments, multiplex amplification is performed such that some or all of the target sequences are amplified within a single reaction vessel. The “plexity” or “plex” of a given multiplex amplification refers generally to the number of different target-specific sequences that are amplified during that single multiplex amplification. In some embodiments, the plexy can be about 12-plex, 24-plex, 48-plex, 96-plex, 192-plex, 384-plex, 768-plex, 1536-plex, 3072-plex, 6144-plex or higher. It is also possible to detect the amplified target sequences by several different methodologies (e.g., gel electrophoresis followed by densitometry, quantitation with a bioanalyzer or quantitative PCR, hybridization with a labeled probe; incorporation of biotinylated primers followed by avidin-enzyme conjugate detection; incorporation of 32P-labeled deoxynucleotide triphosphates into the amplified target sequence).

As used herein, the term “amplification site” refers to a site in or on an array where one or more amplicons can be generated. An amplification site can be further configured to contain, hold, or attach at least one amplicon that is generated at the site.

As used herein, the term “array” refers to a population of sites that can be differentiated from each other according to relative location. Different molecules that are at different sites of an array can be differentiated from each other according to the locations of the sites in the array.

An individual site of an array can include one or more molecules of a particular type. For example, a site can include a single target nucleic acid molecule having a particular sequence or a site can include several nucleic acid molecules having the same sequence (and/or complementary sequence, thereof). The sites of an array can be different features located on the same substrate. Exemplary features include without limitation, droplets, wells in a substrate, beads (or other particles) in or on a substrate, projections from a substrate, ridges on a substrate or channels in a substrate. The sites of an array can be separate substrates each bearing a different molecule. Different molecules attached to separate substrates can be identified according to the locations of the substrates on a surface to which the substrates are associated or according to the locations of the substrates in a liquid or gel. Exemplary arrays in which separate substrates are located on a surface include, without limitation, those having beads in wells.

As used herein, the term “clonal population” refers to a population of nucleic acids that is homogeneous with respect to a particular nucleotide sequence. The homogenous sequence is typically at least 10 nucleotides long, but can be even longer including for example, at least 50, 100, 250, 500 or 1000 nucleotides long. A clonal population can be derived from a single target nucleic acid or template nucleic acid. Typically, all of the nucleic acids in a clonal population will have the same nucleotide sequence. It will be understood that a small number of mutations (e.g., due to amplification artifacts) can occur in a clonal population without departing from clonality.

The term “sensitivity” as used herein is equal to the number of true positives divided by the sum of true positives and false negatives.

The term “specificity” as used herein is equal to the number of true negatives divided by the sum of true negatives and false positives.

As used herein, “providing” in the context of a protein, sample of DNA or RNA, or composition means making the protein, sample of DNA or RNA, or composition, purchasing the protein, sample of DNA or RNA, or composition, or otherwise obtaining the protein, sample of DNA or RNA, or composition.

As used herein, “isolated” refers to material removed from its original environment (e.g., the natural environment if it is naturally occurring), and thus is altered “by the hand of man” from its natural state.

As used herein, the term “each,” when used in reference to a collection of items, is intended to identify an individual item in the collection but does not necessarily refer to every item in the collection unless the context clearly dictates otherwise.

As used in this specification and the appended claims, the term “or” is generally employed in its sense including “and/or” unless the content clearly dictates otherwise. The term “and/or” means one or all of the listed elements or a combination of any two or more of the listed elements. The use of “and/or” in some instances does not imply that the use of “or” in other instances may not mean “and/or.”

Unless otherwise specified, “a,” “an,” “the,” and “at least one” are used interchangeably and mean one or more than one.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure.

As used herein, “have,” “has,” “having,” “include,” “includes,” “including,” “comprise,” “comprises,” “comprising,” or the like are used in their open ended inclusive sense, and generally mean “include, but not limited to, “includes, but not limited to,” or “including, but not limited to.”

It is understood that wherever embodiments are described herein with the language “have,” “has,” “having,” “include,” “includes,” “including,” “comprise,” “comprises,” “comprising,” and the like, otherwise analogous embodiments described in terms of “consisting of” and/or “consisting essentially of” are also provided. The term “consisting of” means including, and limited to, whatever follows the phrase “consisting of.” That is, “consisting of” indicates that the listed elements are required or mandatory, and that no other elements may be present. The term “consisting essentially of” indicates that any elements listed after the phrase are included, and that other elements than those listed may be included provided that those elements do not interfere with or contribute to the activity or action specified in the disclosure for the listed elements.

Conditions that are “suitable” for an event to occur or “suitable” conditions are conditions that do not prevent such events from occurring. Thus, these conditions permit, enhance, facilitate, and/or are conducive to the event.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 4.5, 5, 5.3, and 6. This applies regardless of the breadth of the range.

In the description herein particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements. In several places throughout the application, guidance is provided through lists of examples, which examples can be used in various combinations. In each instance, the recited list serves only as a representative group and should not be interpreted as an exclusive list. It is to be understood that the particular examples, materials, amounts, and procedures are to be interpreted broadly in accordance with the scope and spirit of the invention as set forth herein.

All headings throughout are for the convenience of the reader and should not be used to limit the meaning of the text that follows the heading, unless so specified. The above summary of the present disclosure provided above is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTIONS OF THE FIGURES

FIGS. 1A and 1B. Overview of the enzymatic methods described herein for preventing the detection of false positive uracil residues. FIG. 1A is a schematic illustrating how library reads containing no false positive uracil residues will be amplified preferentially over library reads containing false-positive uracil residues when a U-intolerant polymerase is employed. FIG. 1B is a schematic illustrating an alternate method in which uracil-excising enzymes, such as USER or UDG, are used to remove uracil residues from library fragments, thus preventing the incorporation of the uracil residue into the eventual library read.

FIG. 2. Binomial model predicting the prevalence of false positive uracil residues. Using different off-target C>U conversion rates, the proportion of library fragments containing at least one uracil is plotted at different fragment lengths.

FIG. 3. Workflow diagram illustrating how false positive uracil bases can be removed by carrying out enzymatic deamination and uracil excision prior to library preparation.

FIG. 4. Observed global CG methylations levels with alternative uracil discrimination workflows. Global methylation levels for different genomes when the assay was performed with a Uracil tolerant (+) polymerase, a Uracil intolerant (−) polymerase, or both USER treatment and a Uracil (−) polymerase.

FIG. 5. Library complexity for libraries treated with different uracil discrimination strategies. Uracil (+) and Uracil (−) refers to uracil tolerant and intolerant polymerases, respectively.

FIG. 6. Lambda global methylation values for alternative polymerases in the methylation assay. The lambda control genome is fully unmethylated and methylation signal is indicative of false positives.

FIGS. 7A-7D. Sequencing metrics comparing the performance of Q5 and Q5U. FIG. 7A shows false positive rate observed in the lambda genome in this experiment. FIG. 7B presents duplicate reads. FIG. 7C presents average autosomal coverage over the human genome. FIG. 7D presents median average deviation (MAD) of coverage.

FIG. 8. Correlation of regional methylation levels of CpG Islands with EM-Seq data.

FIG. 9. Visualization of DMRs been identified by EMSeq and the APOBEC assay with different polymerases.

FIGS. 10A and 10B. Workflow diagram illustrating a method involving a single extension with a uracil-intolerant polymerase to create a copy of the original library fragment, followed by cleaving or destroying the original library fragment using a targeted endonuclease, and then followed by PCR using a uracil-tolerant or uracil-intolerant polymerase. FIG. 10A utilizes Fpg/OGG or Endo V. FIG. 10B utilizes lambda exonuclease or T7 exonuclease. For lambda exonuclease, the original library 5′ adapter should be marked with a 5′ phosphate to facilitate degradation. For T7 exonuclease, the primer for the U intolerant polymerase should contain phosphorothioate linkages to confer stability

### DETAILED DESCRIPTION OF ILLUSTRATIVE EMBODIMENTS

The methods described herein utilize enzymes that discriminate against uracil residues, such as for example, uracil-intolerant polymerases, uracil DNA glycosylase (UDG), and/or USER™ (Uracil-Specific Excision Reagent) enzyme, to remove false positive uracil residues from cytidine deaminase mediated methylation sequencing assays. Cytidine deaminases catalyze the deamination of cytosine bases. For example, a class of cytidine deaminases, APOBEC3A, can deaminate cytosine to uracil and methylcytosine to thymine (Schutsky et al., 2017, Nucleic Acids Research; 45 (13): 7655-65). Protein engineering of APOBEC3A has resulted in mutant APOBEC proteins with selectivity towards deamination of 5mC with reduced activity towards deamination of C, however residual activity for deamination of C remains. These false positive conversions by APOBEC enzymes result in conversion into uracil (U), while true positive conversions result in the conversion of 5mC to T. Examples of such engineered APOBEC3A cytidine deaminases include, for example, any of those described in International Patent Application No. PCT/US2023/017846 (“Altered Cytidine Deaminases and Methods of Use”), filed Apr. 7, 2023, which is hereby incorporated by reference in its entirety.

Treatment with Uracil-Intolerant Polymerases

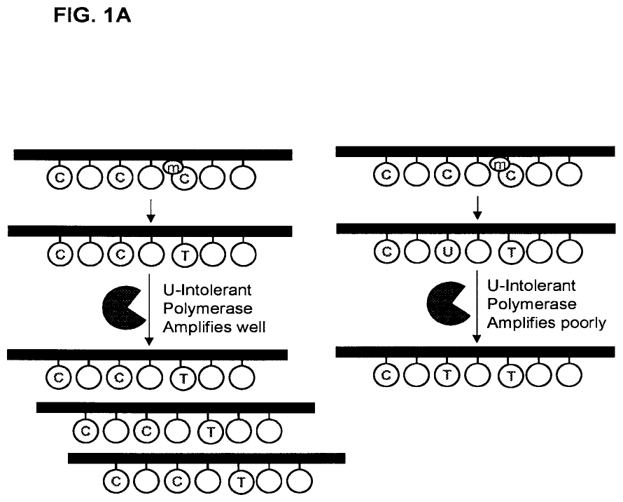

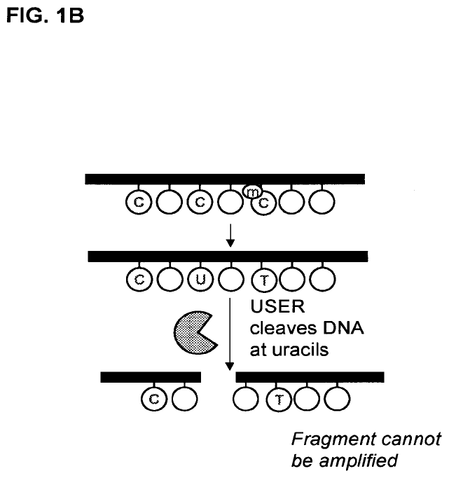

In some embodiments of the methods described herein, the problem of false positive conversions of cytosines to uracils in cytosine deaminase-mediated methylation detection assays is addressed by the utilization of uracil-intolerant polymerases in order to reduce the likelihood that such false positive conversions are detected in the final sequenced library. A schematic illustrating this Reducing Uracils By polymerase (RUBY) method is shown in FIG. 1A. Briefly, with the RUBY methods described herein, after treatment of a preparation of DNA fragments from an input sample with a cytosine deaminase to deaminate 5-methylcytosine (5mC) and/or 5-hydroxymethylcytosine (5hmC) residues and possibly including one or more off-target conversions of a cytosine to an uracil, the preparation of DNA fragments is subject to at least one round of extension with a uracil-intolerant polymerase to selectively extend reads that lack false positive C>U deamination events (FIG. 1A).

In some embodiments, the preparation of DNA fragments is subject to an initial round of extension with a uracil-intolerant polymerase. The preparation of DNA fragments may then be subject to further rounds of extension with a uracil-intolerant polymerase or a uracil-tolerant polymerase. In some embodiments, after an initial round of extension with a uracil-intolerant polymerase, the preparation of DNA fragments may be subject to polymerases chain reaction (PCR) amplification with either a uracil-intolerant polymerase or a uracil-tolerant polymerase.

In some embodiments, a fragmented dsDNA sample may be first subjected to adapter ligation-based library preparation conditions in order to facilitate downstream next generation sequencing (NGS). Such dsDNA samples can include, for example, cfDNA, FFPE or high quality gDNA samples. After adapter ligation, the DNA sample may be treated with an engineered APOBEC enzyme to facilitate selective mC>T deamination. Importantly, with the RUBY method described herein, a single C>U deamination event within a library fragment will disfavor amplification of the entire fragment.

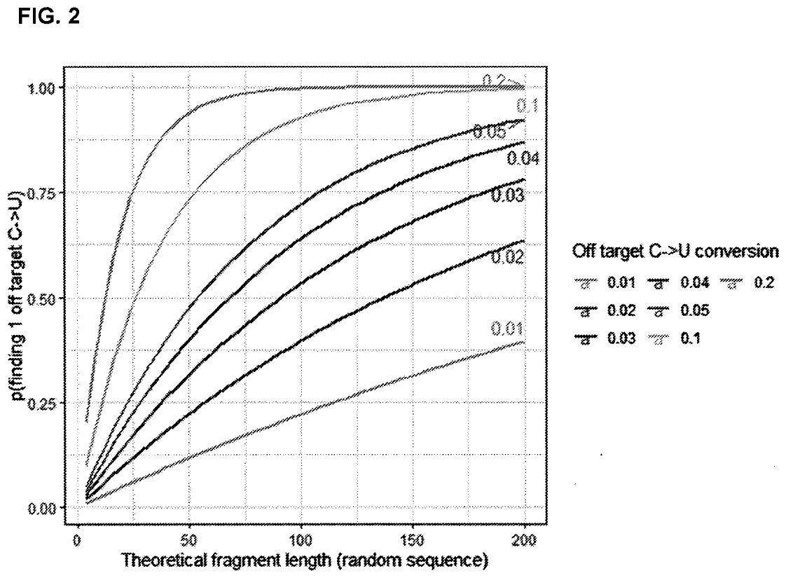

The probability that a fragment will contain a C>U deamination event depends on the rate of off-target cytosine deamination using a given APOBEC enzyme and reaction conditions. Using a binomial model, the probability that a random DNA fragment of a given length (in nucleotides) contains a single C>U event has been calculated, using a range of off-target deamination rates (FIG. 2). Based on this model, low levels of false-positive deamination can be tolerated while still preserving much of the library complexity. Furthermore, fragment length may be used to improve performance by minimizing the probability that a fragment contains a FP event. Loss of library fragments may be particularly severe in regions that have high GC content, due to the higher density of C residues that may be erroneously converted into uracils. Therefore, minimizing the baseline C>U deamination rate will provide optimal performance of this strategy.

In some embodiments of the methods described herein, in addition to extension with a uracil-intolerant polymerases, a preparation of DNA fragments may also be subject to treatment with uracil DNA glycosylase (UDG), an endonuclease, and/or USER™ enzyme to further increase the stringency of uracil discrimination/removal. Treatment with UDG, an endonuclease, and/or USER™ may be before or after extension with a uracil-intolerant polymerase.

In order to modulate the level of uracil discrimination and coverage uniformity in the assay, any of many strategies can be employed. For example, optimization of the deaminase construct and deamination reaction conditions can be used to minimize the amount of off-target C>U conversion. Additionally, different uracil intolerant polymerases having different levels of discrimination and different amplification biases may be used. Uracil intolerant polymerases can be utilized in mixtures with uracil-tolerant polymerases in order to modulate the level of discrimination against uracil residues and optimize coverage uniformity.

### DNA Polymerases

B-family polymerases, commonly used for the PCR amplification, are known to exhibit “uracil read-ahead” function which causes stalling of the polymerase at uracil residues (Greagg et al., 1999, PNAS; 96 (16): 9045-50). Specifically, archaeal B-family polymerases including, but not limited to, those from organisms Pyrococcus furiosus (Pfu), Thermococcus kodakarensis (KOD), Thermococcus litoralis (Tli/Vent), Pyrococcus woesei (Pwo), and Thermococcus fumicolans (Tfu) contain this functionality. The skilled person can easily identify members of each of the subfamilies by using a publicly available database such as the Protein database available at the National Center for Biotechnology Information. Structural and sequence motifs responsible for such uracil read-ahead functionality have been reported. See, for example, published U.S. application No. 20060057682A1; Wardle et al., 2008, Nucleic Acids Research; 36 (3): 705-11; Kropp et al., 2021, ChemBioChem; 22 (21): 3060-66; and Fogg et al., 2002, Nat Struct Biol; 9:922-92.

Due to the uracil-intolerance of these enzymes, existing methylation Next Generation Sequencing (NGS) assays in common use typically employ uracil-tolerant (U-tolerant) polymerases to accommodate amplification of templates containing uracil residues. Examples of U-tolerant polymerases include that are commercially available, but are not limited to, exonuclease-deficient Taq polymerase, KapaU polymerase (Roche), Q5U polymerase (New England Biolabs), and Phusion U polymerase (Fisher Scientific). With U-intolerant polymerases, DNA synthesis will be stopped if a uracil is detected in DNA. Examples of commercially available U-intolerant polymerases include, but are not limited to, Kapa HiFi polymerase (Roche), Ultra II Q5 polymerase (NEB), and Phusion HiFi polymerase (Fisher Scientific).

Any of the many protocols available for the synthesis of a complementary second DNA strand are compatible with the methods described herein. A DNA polymerase, a mixture of all four deoxyribonucleoside 5′-triphosphates (dNTPs), and an appropriate primer are provided for the synthesis of the second complementary strand. These four types of dNTP include adenine (dATP), cytosine (dCTP), guanine (dGTP), and thymine (dTTP). Primers include, but are not limited to, a primer complementary to the 3′ end library adapter, and random oligonucleotides of about 18 to 22 bases in length.

### Degradation at Uracil Residues

In some embodiments, an alternative strategy for reducing the detection of false positive uracil residues utilizes enzymes to specifically cleave library fragments at uracil residues, such as, for example, Uracil DNA Glycosylase (UDG), an endonuclease, such as Endonuclease IV or AP Endonuclease I, and/or an USER™ enzyme cocktail, thus preventing the incorporation of the uracil residue into the eventual library read. Such a method is shown in FIG. 1B.

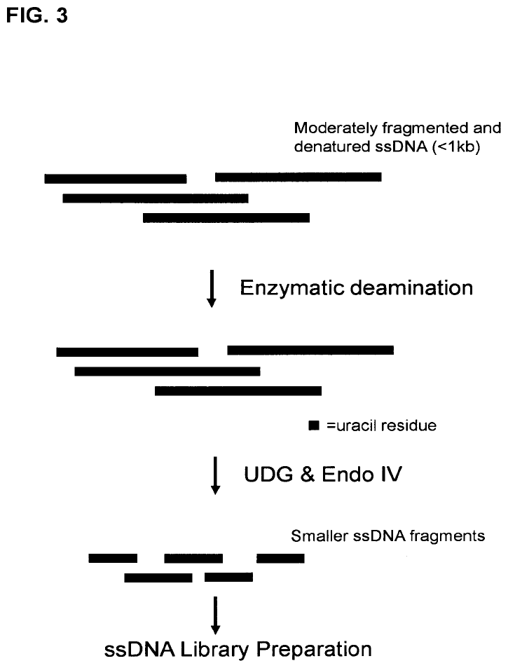

Some embodiments of the methods described herein utilize removing false positive uracil residues prior to sequencing by subjecting a preparation of DNA fragments to enzymatic deamination first, followed by library preparation (FIG. 3). First, input DNA may be minimally fragmented to generate fragments of less than about 1 kb to facilitate proper enzymatic deamination, because long fragments may not be good substrates for this reaction. This fragmented DNA is then subjected to denaturation to obtain a preparation of single stranded DNA (ssDNA) fragments which is then subjected to enzymatic deamination with a cytidine deaminase, including an engineered cytidine deaminase. Then, the ssDNA fragments are treated with Uracil DNA glycosylase (UDG/UNG) and an endonuclease such as Endonuclease IV or AP Endonuclease I.

Alternatively, the ssDNA fragments may be treated with USER™ (Uracil-Specific Excision Reagent) Enzyme, which is a mixture of E. coli uracil DNA glycosylase and the DNA glycosylase-lyase Endonuclease VIII. USER™ Enzyme combines these two enzymatic activities to generate a single nucleotide gap at the location of a uracil residue. UDG catalyzes the excision of a uracil base, forming an abasic (apyrimidinic) site while leaving the phosphodiester backbone intact and the lyase activity of Endonuclease VIII breaks the phosphodiester backbone at the 3′ and 5′ sides of the abasic site so that base-free deoxyribose is released. Examples of USER™ Enzyme include Thermolabile USER II Enzyme (NEB #M5508) and Thermostable USER III Enzyme (NEB #M5509).

Treatment with UDG and an endonuclease or with USER™ Enzyme results in the removal of uracil residues and cleavage of the phosphodiester backbone to generate single stranded fragments depleted of uracil residues which can then serve as substrates to single stranded library preparation methods. Single stranded library preparation methods are well known in the art and include ligation-based approaches (Troll et al., 2019, BMC Genomics; 20 (1): 1-14; and Raine et al., 2017, Nucleic Acids Research; 45 (6): e36) and commercial kits (xGen Methyl-Sequencing DNA Library Prep Kit and Adaptase, Integrated DNA Technologies).

### Degradation of Original Library Strand

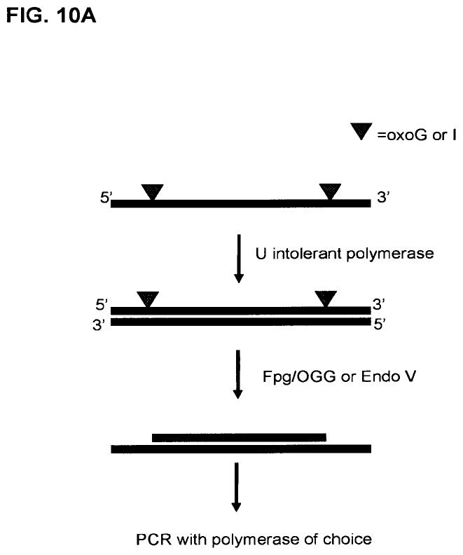

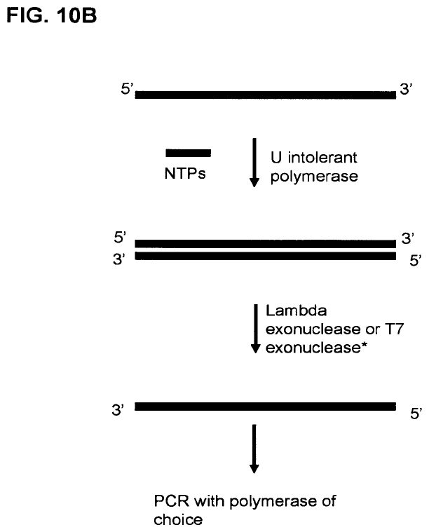

In some embodiments of the methods described herein, a preparation of DNA library fragments from an input sample that has been treated with a cytosine deaminase to deaminate 5-methylcytosine (5mC) and/or 5-hydroxymethylcytosine (5hmC) residues and possibly including one or more off-target conversions of a cytosine to an uracil is subject to a single extension with a U-intolerant polymerase to create a copy of the original library fragment. As the U-intolerant polymerase stalls at any uracil residues present in the original library fragments, complete copies of the original library fragments, with intact adapter 5′ and 3′ adapters are obtained only from original fragments without uracil residues. The original library fragments are then cleaved or destroyed using a targeted endonuclease. PCR amplification is then carried out on the remaining copies of the original library fragments. A U-tolerant, U-intolerant, or other enzyme of choice may be used for PCR amplification. FIGS. 10A and 10B illustrates this method involving a single extension with a uracil-intolerant polymerase to create a copy of the original library fragment, followed by cleaving or destroying the original library fragment using a targeted endonuclease, which is then followed by PCR amplification.

Various approaches may be employed to cleave or destroying the original library fragment. For example, in some embodiments, inosine or 8-oxoguanine residues can be incorporated into the original ligation adapters. Following generation of the second strand with a U intolerant polymerase, the presence of the 8-oxoguanine or inosine residues enables the selective cleavage of the adapter sequences from the original library fragments. Formamidopyrimidine-DNA glycosylase (FPG) or oxoguanine glycosylase (OGG) can be used for the cleavage of adapters sequences containing 8-oxoguanine residues (see, for example, Murphy and George, 2005, Biochem Biophys Res Commun; 329 (3): 869-872; and Murphy and Guo, 2010, Biochem Biophys Res Commun; 392 (3): 335-339) and Endonuclease V can be used for the cleavage of adapters sequences containing inosine residues (see, for example, Cao, 2013, Cell Mol Life Sci; 70 (17): 3145-56). In some embodiments, a 5′ phosphate can be incorporated into the original library fragment, thus marking it for degradation by lambda exonuclease. And, in some embodiments, a primer containing phosphorothioate bonds can be used for second strand generation with the U intolerant polymerase, thus conferring protection to that strand. Treatment with T7 exonuclease will preferentially degrade the original library fragment.

### Uracil De-Glycosylation

With the methods described herein, preparations of double-stranded DNA (dsDNA) fragments or single stranded DNA (ssDNA) fragments may be contacted with an Uracil-DNA-glycosylase. Uracil-DNA-glycosylase (UDG), also known as Uracil-N-glycosylase (UNG), is a highly conserved repair enzyme that catalyzes the excision of uracil from uracil-containing single- and double-stranded DNA but is inactive on RNA. It is a monomeric protein with relatively stable physicochemical properties, a small molecular weight of 25KDa, and is widely present in various prokaryotic and eukaryotic organisms. See, for example, Hölz et al., 2019, Scientific Reports; 9:17822; Schormann et al., 2014, Protein Sci; 23:1667-1685; Zharkov et al., 2010, Mutation Research 685, 11-20; Stivers et al., 2001, Arch Biochem Biophys; 396, 1-9; Parikh et al., 2000, Proc Natl Acad Sci USA; 97:5083; Pearl, 2000, Mutat Res 460, 165-181; Lindahl, 1982, Annu Rev Biochem; 51:61-87; and Lindahl et al., 1977, J Biol Chem; 252:3286-3294.

UDG excises uracil from DNA by hydrolyzing the N-glycoside bond between the uracil base and the sugar-phosphate backbone in single- and double-stranded DNA (Bellamy et al., 2007, Nucleic Acids Res; 35:1478-1487; Slupphaug et al., 1996, Nature 384, 87-92; Stivers et al., 1999, Biochemistry; 38:952-963; and Parikh et al., 2000, Mutat Res; 460:183-199), resulting in the formation of an abasic site (AP-site) having a hemiacetal formation.

A variety of UDG enzymes are commercially available, including, for example, E. coli Uracil-DNA Glycosylase (UDG) (New England Biolabs, Catalog #M0280S, see the worldwide web at neb.com/products/m0280-uracil-dna-glycosylase-udg #Product % 20Information) and a heat-labile Uracil DNA Glycosylase (UDG/UNG) isolated from a psychrophilic marine bacteria (Yeasen Biotechnology (Shanghai) Co., Ltd., Catalog #10707ES, see the worldwide web at yeasenbiotech.com/solutiondetail/79?gclid-EAIaIQobChMI_Oie4unY-gIV3xCtBh0hRwGHEAAYASAAEgKsx_D_BwE). In some embodiments, the UDG is of commercial origin.

Reaction conditions suitable for the UDG-mediated excision of uracil from DNA include, but are not limited to, concentration of the single stranded or double stranded DNA substrate, pH, temperature of the reaction, time of the reaction, and concentration of the UDG enzyme. It is expected that a UDG can function in essentially any buffer. An example of a useful buffer includes, but is not limited to, 1× UDG Reaction Buffer (New England Biolabs, Catalog #B0280S, see the worldwide web at neb.com/products/m0280-uracil-dna-glycosylase-udg #Product % 20Information) which is 20 mM Tris-HCl, 1 mM DTT, 1 mM EDTA (pH 8 at 25° C.). Uracil-DNA Glycosylase is active over a broad pH range, with an optimum at pH 8.0, does not require a divalent cation, and is inhibited by high ionic strength (>200 μM). Uracil-DNA Glycosylase is active in a temperature of 25° C. to 37° C. and in some embodiments the reaction can proceed in a temperature of 25° C. to 37° C. In some embodiments, the reaction can proceed at 37° C. In some embodiments, the reaction can proceed for about 5 minutes, about 10 minutes, about 15 minutes, about 20 minutes, about 30 minutes, about 45 minutes, about 60 minutes, about 90 minutes, about 120 minutes, or any range thereof. In some embodiments, a reaction can include about 0.001 U/μl to about 1 U/μl UDG enzyme, wherein one unit is defined as the amount of enzyme that catalyzes the release of 60 pmol of uracil per minute from double-stranded, uracil-containing DNA. Activity is measured by release of [3H]-uracil in a 50 μl reaction containing 0.2 μg DNA (10⁴-10⁵ cpm/μg) in 30 minutes at 37° C. (see the worldwide web at neb.com/products/m0280-uracil-dna-glycosylase-udg #Product % 20Information). In some embodiments, a reaction can include about 0.05 U/μl UDG. In some embodiments, a reaction can include nucleic acids at a concentration of about 1 ng to about 1 μg of input nucleic acid. In some embodiments, a reaction can include nucleic acids at a concentration of about ˜10 pM to about 200 nM. In some embodiments, a reaction can include nucleic acids at a concentration of about 200 pM to about 20 nM.

With the methods described herein, the target nucleic acids, also referred to herein as “DNA fragments” or “a preparation of DNA fragments from an input sample,” may be essentially any nucleic acid of known or unknown sequence.

Such target nucleic acids are typically derived from primary nucleic acids present in a sample, such as a biological sample. The primary nucleic acids may originate as DNA or RNA. DNA primary nucleic acids may originate in double-stranded DNA (dsDNA) form (e.g., genomic DNA, genomic DNA fragments, cell-free DNA, and the like) from a sample or may originate in single-stranded form from a sample. RNA primary nucleic acids may be mRNA or non-coding RNA, e.g., microRNA or small interfering RNA. A preparation of DNA fragments from an input sample may be single or double stranded DNA.

The primary nucleic acid molecules may represent the entire genetic complement of an organism, e.g., genomic DNA molecules which include both intron and exon sequences, as well as non-coding regulatory sequences such as promoter and enhancer sequences. The primary nucleic acid molecules may represent the entire genetic complement of specific cells of an organism, e.g., from tumor cells, where the genomic DNA molecules which include both intron and exon sequences, as well as non-coding regulatory sequences such as promoter and enhancer sequences. In one embodiment, particular subsets of genomic DNA can be used, such as, for example, particular chromosomes, DNA associated with open chromatin, DNA associated with closed chromatin, or one or more specific sequences such as a region of a specific gene (e.g., targeted sequencing). In one or more embodiments, the primary nucleic acid molecules may represent a particular subset of DNA, e.g., DNA having a specific sequence that anneals with a primer such as one used for targeted sequencing or target enrichment. In one embodiment, a particular subset of DNA can be used, such as cell-free DNA, which can include DNA of the subject including DNA from normal cells, DNA from diseased cells such as tumor cells, and/or DNA from fetal cells.

The primary nucleic acid molecules may represent the entire transcriptome of cells of an organism, e.g., mRNA molecules. The primary nucleic acid molecules may represent the entire transcriptome of specific cells of an organism, e.g., from tumor cells or for instance the cells of a tissue. In one embodiment, the primary nucleic acid molecules may represent a particular subset of mRNA, e.g., mRNA having a specific sequence that anneals with a primer such as one used for targeted sequencing or target enrichment.

A sample, such as a biological sample, can include nucleic acid molecules obtained from biopsies, tumors, scrapings, swabs, blood, mucus, urine, plasma, semen, hair, laser capture micro-dissections, surgical resections, and other clinical or laboratory obtained samples. In some embodiments, the sample can be an epidemiological, agricultural, forensic, or pathogenic sample. In some embodiments, the sample can include cultured cells. In some embodiments, the sample can include nucleic acid molecules obtained from an animal such as a human or mammalian source. In another embodiment, the sample can include nucleic acid molecules obtained from a non-mammalian source such as a plant, bacteria, virus, or fungus. In some embodiments, the source of the nucleic acid molecules may be an archived or extinct sample or species.

Additional non-limiting examples of sources of biological samples can include whole organisms as well as a sample obtained from a subject or a patient. The biological sample can be obtained from any biological fluid or tissue and can be in a variety of forms, including liquid fluid and tissue, solid tissue, and preserved forms such as dried, frozen, and fixed forms. The sample may be of any biological tissue, cells, or fluid. Such samples include, but are not limited to, sputum, blood, serum, plasma, blood cells (e.g., white cells), ascitic fluid, urine, saliva, tears, sputum, vaginal fluid (discharge), washings obtained during a medical procedure (e.g., pelvic or other washings obtained during biopsy, endoscopy or surgery), tissue, nipple aspirate, core or fine needle biopsy samples, cell-containing body fluids, peritoneal fluid, and pleural fluid, or cells therefrom, and free floating nucleic acids such as cell-free circulating DNA. Biological samples may also include sections of tissues such as frozen or fixed sections taken for histological purposes or micro-dissected cells or extracellular parts thereof. In some embodiments, the sample can be a blood sample, such as, for example, a whole blood sample. In another example, the sample is an unprocessed dried blood spot (DBS) sample. In yet another example, the sample is a formalin-fixed paraffin-embedded (FFPE) sample. In yet another example, the sample is a saliva sample. In yet another example, the sample is a dried saliva spot (DSS) sample.

Exemplary biological samples from which target nucleic acids can be derived include, for example, those from a eukaryote, for instance a mammal, such as a rodent, mouse, rat, rabbit, guinea pig, ungulate, horse, sheep, pig, goat, cow, cat, dog, primate, human or non-human primate; a plant, such as Arabidopsis thaliana, corn, sorghum, oat, wheat, rice, canola, or soybean; an algae, such as Chlamydomonas reinhardtii; a nematode such as Caenorhabditis elegans; an insect, such as Drosophila melanogaster, mosquito, fruit fly, honey bee or spider; a fish, such as zebrafish; a reptile; an amphibian, such as a frog or Xenopus laevis; a Dictyostelium discoideum; a fungi, such as Pneumocystis carinii, Takifugu rubripes, yeast, Saccharamoyces cerevisiae, or Schizosaccharomyces pombe; or a protozoan such as Plasmodium falciparum. Target nucleic acids can also be derived from a prokaryote such as a bacterium, Escherichia coli, Staphylococcus or Mycoplasma pneumoniae; an archaeon; a virus such as Hepatitis C virus or human immunodeficiency virus; or a viroid. Target nucleic acids can be derived from a homogeneous culture or population of organisms described herein or alternatively from a collection of several different organisms, for example, in a community or ecosystem.

In some embodiments, a biological sample includes tissue that is processed to obtain the desired primary nucleic acids. In some embodiments, cells are used obtain the desired primary nucleic acids. In some embodiments, nuclei are used to obtain the desired primary nucleic acids. The method can further include dissociating cells, and/or isolating nuclei from cells. Methods for isolating cells and nuclei from tissue are available (WO 2019/236599).

In some embodiments, nucleic acids present in tissue, in cells, or in isolated nuclei can be processed depending on the desired read-out. For instance, nucleic acids can be fixed during processing, and useful fixation methods are available (WO 2019/236599). Fixation can be useful to preserve a sample or maintain contiguity of analytes from a sample, a cell, or a nucleus. Fixation methods preserve and stabilize tissue, cell, and nucleus morphology and architecture, inactivates proteolytic enzymes, strengthens samples, cells, and nuclei so they can withstand further processing and staining, and protects against contamination. Examples of methods where fixation can be useful include, but are not limited to, whole genome sequencing of isolated nuclei and chromosome conformation capture methods such as Hi-C. Common methods of fixation include perfusion, immersion, freezing, and drying (Srinivasan et al., Am J Pathol. 2002 December; 161 (6): 1961-1971.doi: 10.1016/S0002-9440 (10) 64472-0). In some embodiments such as whole genome sequencing, isolated nuclei can be processed to dissociate nucleosomes from DNA while leaving the nuclei intact, and methods for generating nucleosome-free nuclei are available (WO 2018/018008).

In some embodiments, primary nucleic acids in bulk, e.g., from a plurality of cells, can be used to produce a sequencing library as described herein. In other embodiments, individual cells or nuclei can be used as sources of primary nucleic acids to obtain sequence information from single cells and nuclei. Many different single cell library preparation methods are known in the art, including, but not limited to, Drop-seq, Seq-well, and single cell combinatorial indexing (“sci-”) methods. Companies providing single cell products and related technologies include, but are not limited to, Illumina, 10× genomics, Takara Biosciences, BD biosciences, Biorad, 1 cellbio, isoplexis, CellSee, nanoselect, and Dolomite bio. Sci-seq is a methodological framework that employs split-pool barcoding to uniquely label the nucleic acid contents of large numbers of single cells or nuclei. Typically, the number of nuclei or cells can be at least two. The upper limit is dependent on the practical limitations of equipment (e.g., multi-well plates, number of indexes) used in other steps of the methods as described herein. The number of nuclei or cells that can be used is not intended to be limiting and can number in the billions.

The target nucleic acids used in the methods and compositions of the present disclosure can be derived by fragmentation. Random fragmentation refers to the fragmentation of a polynucleotide molecule from a primary nucleic acid sample in a non-ordered fashion by enzymatic, chemical, or mechanical methods. Such fragmentation methods are known in the art and use standard methods (Sambrook and Russell, Molecular Cloning, A Laboratory Manual, third edition). Moreover, random fragmentation is designed to produce fragments irrespective of the sequence identity or position of nucleotides comprising and/or surrounding the break. In one or more embodiments, the random fragmentation is by mechanical means such as nebulization or sonication to produce fragments of about 50 base pairs in length to about 1500 base pairs in length, for example, about 50-700 base pairs in length, about 50-400 base pairs in length. In some preferred embodiments, fragments are about 100 to 300 base pairs in length or about 100 to 200 base pairs in length.

In some embodiments, the DNA fragments are DNA library fragments. Any of the many library preparation protocols available are compatible with the methods described herein. A library may be a whole-genome library or a targeted library. A library includes, but is not limited to, a sequencing library. A multitude of sequencing library methods are known to a skilled person (see, for example, Sequencing Methods Review, available on the world wide web at illumina.com/content/dam/illumina-marketing/documents/products/research reviews/sequencing-methods-review.pdf). For example, library preparation may be for use with any of a variety of next generation sequencing platforms, such as for example, the sequencing by synthesis platform of ILLUMINA® or the ion semiconductor sequencing platform of ION TORRENT™. For example, established ligase-dependent methods or transposon-based methods may be used (see, for example, Head et al, 2014, Biotechniques; 56 (2): 61 and Bruinsma et al., 2019, BMC Genomics; 19:722) and numerous kits for making sequencing libraries by these methods are available commercially from a variety of vendors.

DNA fragments, including DNA library fragments, may be prepared from input sample material such that adapter sequences are ligated to fragments to facilitate downstream workflow steps, such as for example, degradation of the second strand, amplification, and/or sequencing. For example, universal amplification sequences, e.g., sequences present in a universal adaptor, may be placed at the ends of each nucleotide fragment to facilitate amplification. Methods for attaching adapters to a nucleic acid are known to the person skilled in the art. For example, the attachment can be through tagmentation using transposase complexes (WO 2016/130704), or through standard library preparation techniques using ligation (U.S. Pat. Pub. No. 2018/0305753). Addition of an adapter can occur before or after treatment of the target nucleic acid with a cytidine deaminase and/or an uracil de-glycosylase.

Adapter sequences may include 5′ and/or 3′ adapter sequences. An adapter may be attached to just one end of the DNA fragment, for example, 5′ and/or 3′ ends, or to both ends. As used herein, the term “adapter” and its derivatives, e.g., universal adapter, refers generally to any linear oligonucleotide which can be attached to a target nucleic acid. An adapter can be single-stranded or double-stranded DNA or can include both double-stranded and single-stranded regions. An adapter can include a universal sequence that is substantially identical, or substantially complementary, to at least a portion of a primer, for example a universal primer; an index (also referred to herein as a barcode or tag) to assist with downstream error correction, identification, or sequencing; and/or a unique molecular identifier. In some embodiments, the adapter is substantially non-complementary to the 3′ end or the 5′ end of any target sequence present in the sample. In some embodiments, adapter sequences may have one or more phosphorothioate bonds at the 5′ end of the adapter sequences. In some embodiments, suitable adapter lengths are in the range of about 6-100 nucleotides, about 12-60 nucleotides, or about 15-50 nucleotides in length. For instance, The terms “adaptor” and “adapter” are used interchangeably. As used herein, the term “universal,” when used to describe a nucleotide sequence, refers to a region of sequence that is common to two or more nucleic acid molecules where the molecules also have regions of sequence that differ from each other. Non-limiting examples of universal capture sequences include sequences that are identical to or complementary to P5 and P7 primers. The terms “P5” and “P7” may be used when referring to a universal capture sequence or a capture oligonucleotide. The terms “P5” (P5 prime) and “P7” (P7 prime) refer to the reverse complement of P5 and P7, respectively. It will be understood that any suitable universal capture sequence or a capture oligonucleotide can be used in the methods presented herein, and that the use of P5 and P7 are exemplary embodiments only. Uses of capture oligonucleotides such as P5 and P7 or their complements on flowcells are known in the art, as exemplified by the disclosures of WO 2007/010251, WO 2006/064199, WO 2005/065814, WO 2015/106941, WO 1998/044151, and WO 2000/018957. For example, any suitable forward amplification primer, whether immobilized or in solution, can be useful in the methods presented herein for hybridization to a complementary sequence and amplification of a sequence. Similarly, any suitable reverse amplification primer, whether immobilized or in solution, can be useful in the methods presented herein for hybridization to a complementary sequence and amplification of a sequence. One of skill in the art will understand how to design and use primer sequences that are suitable for capture and/or amplification of nucleic acids as presented herein.

DNA fragments, including DNA library fragments, can have an average strand length that is desired or appropriate for a particular application of the methods, compositions, or kits set forth herein. For example, the average strand length can be less than about 100,000 nucleotides, 50,000 nucleotides, 10,000 nucleotides, 5,000 nucleotides, 1,000 nucleotides, 500 nucleotides, 300 nucleotides 200 nucleotides, 100 nucleotides, or 50 nucleotides. Alternatively, or additionally, the average strand length can be greater than about 10 nucleotides, 50 nucleotides, 100 nucleotides, 200 nucleotides, 500 nucleotides, 1,000 nucleotides, 5,000 nucleotides, 10,000 nucleotides, 50,000 nucleotides, or 100,000 nucleotides. The average strand length for a population of DNA fragments can be in a range between any maximum and minimum value set forth above.

In some embodiments, DNA fragments, including DNA library fragments, may be of a shorter length, for example, about 50 nucleotides to about 500 nucleotides in length, about 50 nucleotides to about 300 nucleotides in length, about 50 nucleotides to about 250 nucleotides in length, about 50 nucleotides to about 200 nucleotides in length, about 50 nucleotides to about 100 nucleotides in length, about 100 nucleotides to about 200 nucleotides in length, about 100 nucleotides to about 250 nucleotides in length, about 100 nucleotides to about 300 nucleotides in length, or about 100 nucleotides to about 500 nucleotides in length. Shorter fragment length can be employed to maximize the overall performance of the enzymatic error-correction, by minimizing the number of potential false-positive uracils that may be present in any one individual DNA fragment. By minimizing the number of false positives in each fragment, the probability of successfully repairing library fragments and mediating their downstream amplification may be increased, thus preserving library quality and diversity.

### Cytosine Deaminase

With the methods described herein, a sample including single-stranded DNA (ssDNA) fragments may be contacted with a cytosine deaminase to deaminate methylated cytosines. In some embodiments, a sample including single-stranded DNA (ssDNA) fragments is a preparation of denatured library fragments. In some embodiments, the library fragments may include 5′ and/or 3′ adapter sequences.

As used herein, a “cytidine deaminase enzyme” refers to an enzyme that deaminates cytosine and/or one or more cytosine derivatives. The deamination occurs at the amino group of the C4 position of the cytosine or cytosine derivative. For example, a cytidine deaminase enzyme may deaminate cytosine to form U, may deaminate mC to form T, and/or may deaminate hydroxymethylcytosine (hmC) to form hmU. A nonlimiting example of a cytidine deaminase enzyme that may deaminate cytosine to form U, may deaminate mC to form T, and/or may deaminate hmC to form hmU is apolipoprotein B mRNA editing enzyme, catalytic polypeptide-like (APOBEC). Nonlimiting examples of such APOBECs include APOBEC1, APOBEC2, APOBEC3A, APOBEC3B, APOBEC3C, APOBEC3E, APOBEC3F, APOBEC3G, APOBEC3H, and APOBEC4. As used herein, the term “methylcytosine” or “mC” refers to cytosine that includes a methyl group (—CH3 or—Me). The methyl group may be located at the 5 position of the cytosine, in which case the mC may be referred to as 5mC.

In some embodiments, a cytidine deaminase is an altered cytidine deaminase, recombinantly engineered to include a substitution mutation at one or more residues when compared to a reference cytidine deaminase. An altered cytidine deaminase can be based on a member of the AID subfamily, the APOBEC1 subfamily, the APOBEC2 subfamily, the APOBEC3 subfamily (e.g., the 3A subfamily, the 3B subfamily, the 3C subfamily, the 3D subfamily, the 3F subfamily, the 3G subfamily, or the 3H subfamily), or the APOBEC4 subfamily. The skilled person will readily appreciate that such an altered or engineered cytidine deaminase described herein is not naturally occurring. In some embodiments, such an altered or engineered cytidine deaminase demonstrates selective deamination for mC.

An altered cytidine deaminase may be one of three types of altered cytidine deaminases. One type of altered cytidine deaminase preferentially deaminates 5mC instead of C (i.e., converts 5mC to T at a greater rate than converting C to U) and is referred to herein as having “cytosine-defective deaminase activity.” A second type of altered cytidine deaminase preferentially deaminates C instead of 5mC (i.e., converts C to U at a greater rate than converting 5mC to T) and is referred to herein as having “5mC-defective deaminase activity.” A third type of altered cytidine deaminase preferentially deaminates C and 5mC to U and T, respectively, and has significantly reduced deamination of 5hmC, 5fC, and 5caC. The third type is referred to herein as having “5hmC-defective deaminase activity.” Unless the context indicates otherwise, reference to an altered cytidine deaminase includes altered cytidine deaminases having cytosine-defective deaminase activity, altered cytidine deaminases having 5mC-defective deaminase activity, and altered cytidine deaminases having 5mC-defective deaminase activity.

Altered cytidine deaminases include apolipoprotein B mRNA editing enzymes, catalytic polypeptide-like (APOBEC) and activation induced cytidine deaminase (AID). Wild-type APOBEC and AID cytidine deaminases have the activity of deaminating cytidine (C) of DNA and/or RNA to form uridine (U). An altered cytidine deaminase of the present disclosure has an altered rate of deamination of C, 5mC, and/or 5hmC when compared to the wild-type enzyme. A cytidine deaminase of the present disclosure can be referred to herein as an “altered cytidine deaminase,” “recombinant cytidine deaminase,” “mutant cytosine deaminase,” or “modified cytidine deaminases” and refers to any of the altered cytosine deaminases described herein that comprise one or more changes from the reference (i.e., wildtype) amino acid sequence that provide the unexpected property of an altered deamination profile, e.g., alters its ability to preferentially deaminate one form of cytosine over another.

Whether a protein has cytidine deaminase activity may be determined by in vitro assays. On example of an in vitro assay is based on digestion with the restriction enzyme SwaI. A protein that can deaminate 5mC to thymidine has cytidine deaminase activity.

An altered cytidine deaminase that preferentially deaminates 5mC instead of C (i.e., has cytosine-defective deaminase activity) can have a catalytic efficiency that is at least 10-fold, at least 50-fold, or at least 100-fold higher on 5mC than C substrates. In one embodiment, an altered cytidine deaminase that preferentially deaminates 5mC instead of C can have a catalytic efficiency that is no greater than 1500-fold higher on 5mC than C substrates.

An altered cytidine deaminase that preferentially deaminates C instead of 5mC (i.e., has 5mC-defective deaminase activity) can have a catalytic efficiency that is at least 10-fold, at least 50-fold, or at least 100-fold higher on C than 5mC substrates. In one embodiment, an altered cytidine deaminase that preferentially deaminates C instead of 5mC can have a catalytic efficiency that is no greater than 1500-fold higher on C than 5mC substrates.

When compared to a wild type cytidine deaminase, an altered cytidine deaminase that deaminates C and 5mC to U and T, respectively, and has significantly reduced deamination of 5hmC (i.e., has 5hmC-defective deaminase activity), the deamination of 5hmC by an altered cytidine deaminase disclosed herein is reduced by at least 80%, at least 90%, or at least 99% compared to the wild type cytidine deaminase. In one embodiment, the deamination of 5hmC by an altered cytidine deaminase disclosed herein is undetectable using an assay such as the SwaI-based assay.

In certain embodiments, an altered cytidine deaminase of the present disclosure is based on a member of the APOBEC protein family. An altered cytidine deaminase of the present disclosure that is “based on” a member of the APOBEC protein family means the altered cytidine deaminase is an APOBEC protein that includes one or more of the substitution mutations described herein as compared to a reference APOBEC sequence. An altered cytidine deaminase of the present disclosure that is “based on” a member of the APOBEC protein family can also include conservative and/or nonconservative mutations as described herein.

The APOBEC protein family includes subfamilies AID, APOBEC1, APOBEC2, APOBEC3 (including 3A, 3B, 3C, 3D, 3F, 3G, 3H), and APOBEC4. An altered cytidine deaminase of the present disclosure can be based on a member of the AID subfamily, the APOBEC1 subfamily, the APOBEC2 subfamily, the APOBEC3 subfamily (e.g., the 3A subfamily, the 3B subfamily, the 3C subfamily, the 3D subfamily, the 3F subfamily, the 3G subfamily, or the 3H subfamily), or the APOBEC4 subfamily. An altered cytidine deaminase of the present disclosure can be based on a member of the APOBEC protein family from a vertebrate, such as a mammal. Examples of mammals include, but are not limited to, rodents, primates, rabbit, bovine (e.g., cow), porcine (e.g., pig), and equine (e.g., horse). An example of a primate is a human and a chimpanzee.

The APOBEC protein family is a member of the large cytidine deaminase superfamily that contains a canonical zinc-dependent deaminase (ZDD) signature motif embedded within a core cytidine deaminase fold. This fold includes a five-stranded mixed beta (b)-sheet surrounded by six alpha (a)-helices with the order a1-b1-b2-a2-b3-a3-b4-a4-b5-a5-a6 (Salter et al., 2016, Trends Biochem Sci; 41 (7): 578-594. doi: 10.1016/j.tibs.2016.05.001; Salter et al., 2018, Trends Biochem Sci; 43 (8): 606-622 doi.org/10.1016/j.tibs.2018.04.013). Each cytidine deaminase domain core structure of APOBEC proteins contains a highly conserved spatial arrangement of the catalytic center residues of a zinc-binding motif H-[P/A/V]-E-X_{[23-28]}-P—C-X_{[2-4]}-C(SEQ ID NO: 1) (referred to herein as the ZDD motif, where X is any amino acid, and the subscript range of numbers after X refers to the number of amino acids) (Salter et al., 2016, Trends Biochem Sci; 41 (7): 578-594. doi: 10.1016/j.tibs.2016.05.001). Without intending to be limited by theory, the H and two C residues coordinate a Zn atom, and the E residue polarizes a water molecule near the Zn-atom for catalysis (Chen et al., 2021, Viruses; 13:497).

Some members of the APOBEC protein family, e.g., the AID subfamily, the APOBEC1 subfamily, the APOBEC2 subfamily, the APOBEC3A subfamily, the APOBEC3C subfamily, the APOBEC3H subfamily, and the APOBEC4 subfamily, include one copy of the ZDD motif. Other members of the APOBEC protein family, e.g., the APOBEC3B subfamily, the APOBEC3D subfamily, the APOBEC3F subfamily, and the APOBEC3G subfamily, include two copies of the ZDD motif, but often only the C-terminal copy is active (Salter et al., 2016, Trends Biochem Sci; 41 (7): 578-594. doi: 10.1016/j.tibs.2016.05.001). Thus, an altered cytidine deaminase disclosed herein includes one or two ZDD motifs. In one embodiment, an altered cytidine deaminase based on a member of the APOBEC3A subfamily includes the following ZDD motif: HXEX₂₄SW(S/T)PCX_{[2-4]}CX₆FX₈LX₅R(L/I)YX_{[8-11]}LX2LX_{[10]}M (SEQ ID NO: 2) (where X is any amino acid, and the subscript number or range of numbers after X refers to the number of amino acids) (Salter et al., 2016, Trends Biochem Sci, 41 (7): 578-594).

In one embodiment, an altered cytidine deaminase disclosed herein is a member of the following subfamilies, APOBEC3A, APOBEC3B, APOBEC3C, APOBEC3D, APOBEC3F, and APOBEC3G, and can include one or more highly conserved sites that are part of the active site and within the ZDD motif SEQ ID NO: 1. The sites include tryptophan at position 98 and serine or threonine at position 99 (Kouno et al., 2017, Nat. Comm; 8:15024).

In addition to the ZDD motif, a member of the APOBEC protein family also includes other highly conserved residues that are part of the active site but not present as part of the ZDD motif SEQ ID NO: 1. A member the APOBEC3A subfamily, APOBEC3B subfamily, APOBEC3C subfamily, APOBEC3D subfamily, APOBEC3F subfamily, and APOBEC3G subfamily typically includes one or more of the following highly conserved sites that are part of the active site: arginine at position 28; histidine, asparagine, or arginine at position 29; serine or threonine, preferably threonine, at position 31; asparagine or aspartic acid at position 57; tyrosine or phenylalanine at position 130; asparagine or tyrosine at position 131; asparagine, tyrosine, or phenylalanine, preferably tyrosine, at position 132; and arginine or lysine at position 189 (Kouno et al., 2017, Nat. Comm; 8:15024, DOI: 10.1038/ncomms15024).

An altered cytidine deaminase of the present disclosure includes a substitution mutation at one or more residues when compared to a reference cytidine deaminase. A substitution mutation can be at the same position or a functionally equivalent position compared to the reference cytidine deaminase. Reference cytidine deaminases and functionally equivalent positions are described in detail herein. The skilled person will readily appreciate that an altered cytidine deaminase described herein is not naturally occurring.

A reference cytidine deaminase can be a member of the APOBEC protein family. Essentially any known member of the APOBEC protein family can be a reference cytidine deaminase. The skilled person can easily identify members of each of the subfamilies by using a publicly available database such as the Protein database available at the National Center for Biotechnology Information (ncbi.nlm.nih.gov/protein) and searching for APOBEC1, APOBEC2, APOBEC3A, APOBEC3B, APOBEC3C, APOBEC3D, APOBEC3F, APOBEC3G, APOBEC3H, APOBEC4, or, when identifying members of the AID family, Activation-induced cytidine deaminase. A wild type reference cytidine deaminase has the activity of binding single-stranded DNA (ssDNA) and deaminating a cytosine present on the ssDNA to convert it to uracil.

In one embodiment, a wild type reference cytidine deaminase has the activity of binding single-stranded RNA (ssRNA) and deaminating a cytosine present on the ssRNA to convert it to uracil. Methods for determining whether a protein binds ssDNA or ssRNA and deaminates a cytosine present are known to the skilled person.

In one embodiment, an altered cytidine deaminase has an amino acid sequence that is based on a reference sequence which is a member of the APOBEC protein family includes a ZDD motif H-[P/A/V]-E-X_{[23 28]}-P—C-X_{[2-4}]-C(SEQ ID NO: 1) and at least one substitution mutation disclosed herein. Optionally, an altered cytidine deaminase includes other active site residues disclosed herein. Non-limiting examples of reference cytidine deaminase proteins are shown in the following table.

In one embodiment, an altered cytidine deaminase has an amino acid sequence that is based on a reference sequence that is a member of the APOBEC3A subfamily, and includes a ZDD motif HXEX₂₄SW(S/T)PCX_{[2-4]}CX₆FX₈LX5R(L/I)YX_{[8-11]}LX2LX_{[10]}M (SEQ ID NO: 2) (where X is any amino acid, and the subscript number or range of numbers after X refers to the number of amino acids) and at least one substitution mutation disclosed herein. In one embodiment, the substitution mutation is a substitution mutation at the underlined tyrosine, such as a substitution mutation to alanine (A). Optionally, the altered cytidine deaminase includes other active site residues disclosed herein.

In one embodiment, the amino acid sequence of an altered cytidine deaminase includes the amino acids of a member of the APOBEC3A subfamily: X_{[16-26]}-GRXXTXLCYXV-X₁₅-GXXXN-X₁₂-HAEXXF-X₁₄-YXXTWXXSWSPC-X_{[2-4}]-CA-X₅-FL-X₇-LXIXXXR(L/I)Y-X₈-GLXXLXXXG-X₅-M-X₄-FXXCWXXFV-X₆-FXPW-X₁₃-LXXI-X_{[2-6]} (SEQ ID NO: 3) (where X is any amino acid, and the subscript number or range of numbers after X refers to the number of amino acids), or a subset thereof, and at least one substitution mutation disclosed herein. In one embodiment, the substitution mutation is a substitution mutation at the underlined tyrosine, such as a substitution mutation to alanine (A) or to tryptophan (W).

In one embodiment, the amino acid sequence of an altered cytidine deaminase includes the amino acids of a member of the APOBEC3A subfamily: X₂₆-GRXXTXLCYXV-X₁₅-G-X₁₆-HAEXXF-X₁₄-YXXTWXXSWSPC-X₄-CA-X₅-FL-X₇-LXIFXXR(L/I)Y-X₈-GLXXLXXXG-X₅-M-X₄-FXXCWXXFV-X₆-FXPW-X₁₃-LXXI-X₆ (SEQ ID NO: 4) (where X is any amino acid, and the subscript number after X refers to the number of amino acids present), or a subset thereof, and at least one substitution mutation disclosed herein. In one embodiment, the substitution mutation is a substitution mutation at the underlined tyrosine (Y), such as a substitution mutation to alanine (A) or to tryptophan (W).

A substitution mutation can be at the same position or a functionally equivalent position compared to a reference cytidine deaminase. By “functionally equivalent” it is meant that the altered cytidine deaminase has the amino acid substitution at the amino acid position in a reference cytidine deaminase that has the same functional role in both the reference cytidine deaminase and the altered cytidine deaminase.

In general, functionally equivalent substitution mutations in two or more different cytidine deaminases occur at homologous amino acid positions in the amino acid sequences of the cytidine deaminases. Hence, use herein of the term “functionally equivalent” also encompasses mutations that are “positionally equivalent” or “homologous” to a given mutation, regardless of whether or not the particular function of the mutated amino acid is known. It is possible to identify the locations of functionally equivalent and positionally equivalent amino acid residues in the amino acid sequences of two or more different cytidine deaminases on the basis of sequence alignment and/or molecular modelling. For example, the tyrosine at residue 130 of the APOBEC3A proteins of Homo sapiens, Pongo pygmaeus, Nomascus leucogenys, Pan troglodytes, and Gorilla and the tyrosine at residue 133 of the APOBEC3A protein from Macaca fascicularis are functionally equivalent and positionally equivalent. The skilled person can easily identify functionally equivalent residues in cytidine deaminases.

In one embodiment, an altered cytidine deaminase has an amino acid sequence that is structurally similar to a reference cytidine deaminase disclosed herein. In one embodiment, a reference cytidine deaminase is one that includes the amino acid sequence of a sequence listed in Table 1.

TABLE 1
Examples of members of the APOBEC protein subfamilies.

| APOBEC protein | |
|---|---|
| family | Non-limiting examples |
| AID | UniProt: Q9GZX7; UniProt: G3QLD2; Uniprot Q9WVE0 |
| APOBEC1 | UniProt: P41238; NCBI XP_030856728.1; Uniprot P51908 |
| APOBEC2 | UniProt: Q9Y235; Uniprot G3SGN8; Uniprot Q9WV35 |
| APOBEC3A | UniProt: P31941 (SEQ ID NO: 12); GenBank: XP_045219544.1 |
| | GenBank: AER45717.1; GenBank: XP_003264816.1; GenBank: |
| | PNI48846.1; GenBank: ADO85886.1 |
| APOBEC3B | UniProt: Q9UH17; Uniprot G3QV16; Uniprot F6M3K5 |
| APOBEC3C | UniProt: Q9NRW3; Uniprot Q694B5; Uniprot B0LW74 |
| APOBEC3D | UniProt: Q96AK3; NCBI NP_001332895.1; NCBI |
| | NP_001332931.1 |
| APOBEC3F | UniProt: Q8IUX4; Uniprot G3RD21; Uniprot Q1G0Z6 |
| APOBEC3G | UniProt: Q9HC16; Uniprot Q694C1; Uniprot U5NDB3 |
| APOBEC3H | UniProt: Q6NTF7; Uniprot B7T0U7; Uniprot Q19Q52 |
| APOBEC4 | UniProt: Q8WW27; NCBI XP_004028087.1; Uniprot Q497M3 |

UniProt, database of protein sequence and functional information, available at uniprot.org; GenBank, collection of nucleotide sequences and their protein translations, available at ncbi.nlm.nih.gov/protein/.

As used herein, an altered cytidine deaminase may be “structurally similar” to a reference cytidine deaminase if the amino acid sequence of the altered cytidine deaminase possesses a specified amount of sequence similarity and/or sequence identity compared to the reference cytidine deaminase.

Structural similarity of two amino acid sequences can be determined by aligning the residues of the two sequences (for example, a candidate altered cytidine deaminase and a reference cytidine deaminase described herein) to optimize the number of identical amino acids along the lengths of their sequences; gaps in either or both sequences are permitted in making the alignment in order to optimize the number of identical amino acids, although the amino acids in each sequence must nonetheless remain in their proper order. A candidate altered cytidine deaminase is the cytidine deaminase being compared to the reference cytidine deaminase. A candidate altered cytidine deaminase that has structural similarity with a reference cytidine deaminase and cytidine deaminase activity is an altered cytidine deaminase.

Unless modified as otherwise described herein, a pair-wise comparison analysis of amino acid sequences can be conducted, for instance, by the local homology algorithm of Smith and Waterman, 1981, Adv. Appl. Math; 2:482, by the homology alignment algorithm of Needleman and Wunsch, 1907, J Mol Biol; 48:443, by the search for similarity method of Pearson and Lipman, 1988, Proc Nat'l Acad Sci USA; 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Current Protocols in Molecular Biology, Ausubel et al., eds., Current Protocols, a joint venture between Greene Publishing Associates, Inc. and John Wiley & Sons, Inc., supplemented through 2004). One example of an algorithm that is suitable for determining structural similarity is the BLAST® algorithm, which is described in Altschul et al., 1990, J Mol Biol; 215:403-410. The BLAST® algorithm can be used to calculate percent sequence identity and percent sequence similarity between two sequences. Software for performing BLAST® analyses is publicly available through the National Center for Biotechnology Information.

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions. Thus, in one embodiment the amino acid sequence of a cytidine deaminase protein having sequence similarity to a reference sequence may include conservative substitutions of amino acids present in that reference sequence.

A conservative substitution for an amino acid in a protein may be selected from other members of the class to which the amino acid belongs. For example, it is well-known in the art of protein biochemistry that an amino acid belonging to a grouping of amino acids having a particular size or characteristic (such as charge, hydrophobicity, or hydrophilicity) can be substituted for another amino acid without altering the activity of a protein, particularly in regions of the protein that are not directly associated with biological activity. For example, amino acids having a non-polar side chain include alanine, glycine, isoleucine, leucine, methionine, phenylalanine, proline, tryptophan, and valine; amino acids having a hydrophobic side chain include glycine, alanine, valine, leucine, isoleucine, proline, phenylalanine, methionine, and tryptophan; amino acids having a polar side chain include arginine, asparagine, aspartic acid, glutamine, glutamic acid, histidine, lysine, serine, cysteine, tyrosine, and threonine; and amino acids having an uncharged side chain include glycine, serine, cysteine, asparagine, glutamine, tyrosine, and threonine.

Thus, as used herein, reference to a cytidine deaminase as described herein, such as reference to the amino acid sequence of one or more SEQ ID NOs described herein can include a protein with at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence similarity to the reference cytidine deaminase. Examples of altered cytidine deaminases having similarity with a reference amino acid sequence includes those having, for instance, at least 80%, at least 85%, at least 90%, or at least 95% similarity with SEQ ID NO: 5 and having an alanine at amino acid 130. Other examples of altered cytidine deaminases having similarity with a reference amino acid sequence includes those having, for instance, at least 80%, at least 85%, at least 90%, or at least 95% similarity or identity with SEQ ID NO: 6 and having an alanine at amino acid 130 and a histidine at amino acid 132.

Alternatively, as used herein, reference to a cytidine deaminase as described herein, such as reference to the amino acid sequence of one or more SEQ ID NOs described herein can include a protein with at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to the reference cytidine deaminase. Examples of altered cytidine deaminases having identity with a reference amino acid sequence includes those having, for instance, at least 80%, at least 85%, at least 90%, or at least 95% similarity with SEQ ID NO: 5 and having an alanine (A) at amino acid 130. Other examples of altered cytidine deaminases having identity with a reference amino acid sequence includes those having, for instance, at least 80%, at least 85%, at least 90%, or at least 95% similarity or identity with SEQ ID NO: 6 and having an alanine (A) at amino acid 130 and a histidine (H) at amino acid 132.

An altered cytidine deaminase of the present disclosure may include a substitution mutation at a position functionally equivalent to tyrosine at position 130 (Y130) in a member of the APOBEC3A subfamily. Accordingly, an alignment can be produced using a member of the APOBEC3A subfamily and another candidate altered cytidine deaminase from the APOBEC3A subfamily or a different APOBEC subfamily. In one embodiment, the candidate is selected from APOPEC subfamilies APOBEC1 or AID. An example of an algorithm that can be used to produce an alignment is Clustal O. In some APOBEC family proteins, the wild type residue at a position functionally equivalent to Y130 is phenylalanine (F).

In another embodiment, an altered cytidine deaminase of the present disclosure includes a substitution mutation at a position functionally equivalent to the tyrosine (Y) of ZDD motif HXEX₂₄SW(S/T)PCX_{[2-4]}CX₆FX₈LX5R(L/I)YX_{[8-11]}LX2LX_{[10]}M (SEQ ID NO: 2) in a member of the APOBEC family, such as a member of the APOBEC3A subfamily. The underlined tyrosine (Y) of SEQ ID NO: 2 is the position functionally equivalent to the tyrosine amino acid 130 of the wild type APOBEC3A protein (SEQ ID NO: 12).

In one embodiment, the substitution mutation at a position functionally equivalent to Y130 increases cytidine deaminase activity and preferentially acts on 5mC compared to cytosine (i.e., has cytosine-defective deaminase activity). The substitution mutation can be a mutation to alanine (A), glycine (G), phenylalanine (F), histidine (H), glutamine (Q), methionine (M), asparagine (N), lysine (K), valine (V), aspartic acid (D), glutamic acid (E), serine(S), cysteine (C), proline (P), or threonine (T). For example, the altered cytidine deaminase can comprise SEQ ID NO: 9, wherein X is selected from A, G, F, H, Q, M, N, K, V, D, E, S, C, P or T (and is not Y), or can comprise SEQ ID NO: 10, wherein Z is selected from A, G, F, H, Q, M, N, K, V, D, E, S, C, P or T (and is not Y), preferably, in one embodiment, X or Z is A or L. In an exemplary aspect of this embodiment, the substitution mutation at a position functionally equivalent to Y130 is a mutation to alanine (A), (e.g., SEQ ID NO: 5). Specific examples of altered cytidine deaminases having increased activity and preferentially acting on 5mC compared to cytosine include SEQ ID NO: 5 or a sequence having at least 90%, at least 95%, at least 98%, at least 99% sequence identity to SEQ ID NO: 5 and comprising Y130A.

An altered cytidine deaminase of the present disclosure having cytosine-defective deaminase activity (i.e., converts 5mC to T at a greater rate than converting C to U) optionally includes a second substitution mutation at a position two, three, four, or five amino acids on the C-terminal side of the Y130 position, or functionally equivalent to the Y130 position. In one embodiment, the second mutation is a tyrosine (Y), tryptophan (W), cysteine (C), histidine (H), or phenylalanine (F) at a position two, three, four, or five amino acids on the C-terminal side of the Y130 position, or functionally equivalent to the Y130 position. In one embodiment, the second mutation is at a position functionally equivalent to tyrosine at position 132 (Y132) in a member of the APOBEC3A subfamily. An APOBEC protein, such as an APOBEC3A protein, containing substitution mutations at both the first site, a position functionally equivalent to Y130, and the second site, at a position two, three, four, or five amino acids on the C-terminal side of the Y130 position, increases the preferential activity to act on 5mC compared to the same APOBEC protein, such as an APOBEC3A protein, containing one substitution mutation at Y130. In one embodiment, the substitution mutation at the second position is an amino acid having a positively charged side chain and selected from arginine (R), histidine (H), lysine (L), or a polar side chain selected from glutamine (Q). In one embodiment, the substitution mutation at the second position is histidine (H), such as Y132 to histidine. The double mutant containing both first and second mutations can be any substitution mutation at a position functionally equivalent to Y130 described herein and any second substitution mutation at a position two, three, four, or five amino acids on the C-terminal side of the Y130 position described herein, in any combination. For example, the altered cytidine deaminase can be, for example, SEQ ID NO: 4 and have a substitution at Y130 and Y132, or the position functionally equivalent to Y130 and Y132 as described herein. One example of an altered cytidine deaminase is SEQ ID NO: 11 comprising Y130X and Y132Z, where X is selected from (A), (L), or (W) (preferably (A)), and Z is selected from (R), (H), (L), or (Q), preferably (H). This encompasses examples including, but not limited to, for example Y130A and Y132R, Y130A and Y132H, Y130A and Y132L, Y130A and Y132Q, Y130L and Y132R, Y130L and Y132H, Y130L and Y132L, Y130L and Y132Q, Y130W and Y132R, Y130W and Y132H, Y130W and Y132L, Y130W and Y130Q, or any suitable combinations therein. In one embodiment, the double mutant includes substitution mutations Y130A and Y132H. Specific examples of altered cytidine deaminases having both substitution mutations and preferentially acting on 5mC compared to the APOBEC protein having just the single substitution mutation at cytosine include SEQ ID NO: 6 or a sequence having at least 90%, at least 95%, at least 98%, at least 99% sequence identity to SEQ ID NO: 6 and comprising Y130A and Y132H.

The person of ordinary skill in the art can confirm the 5mC preferential deaminase activity of the arginine, glutamine, histidine, and lysine substitution mutations at the second position in the double mutants described above. For example, double mutants can be constructed to create an altered cytidine deaminase having a first substitution mutation at a position functionally equivalent to Y130 and a second arginine, glutamine, histidine, or lysine substitution mutation at the tyrosine position two amino acids on the C-terminal side of the Y130 position, and then evaluated for deamination of C residues in one assay and deamination of 5mC residues in a second assay. Using an assay such as the SwaI-based assay described herein, the ratio of 5mC deamination and C deamination can be compared to identify those double mutants that preferentially deaminate 5mC compared to C. One of ordinary skill in the art could similarly test double mutants having a tyrosine at a position three, four or five positions C-terminal to the position functionally equivalent to Y130 and confirm that a substitution mutation at that position to arginine, glutamine, histidine, or lysine, in combination with a mutation at the position functionally equivalent to Y130 (such as Y130A), as double mutants that preferentially deaminate 5mC compared to C.

Some embodiments presented herein relate to substitution mutations that result in 5mC-defective deaminase activity (i.e., converts C to U at a greater rate than converting 5mC to T). In one embodiment, the substitution mutation at a position functionally equivalent to Y130 increases cytidine deaminase activity and preferentially acts on cytosine compared to 5mC and is a mutation to an amino acid having a non-polar side chain or a hydrophobic side chain, such as leucine (L) or tryptophan (W). In an exemplary aspect of this embodiment, the substitution mutation at a position functionally equivalent to Y130 is a mutation to leucine. Other examples of mutations that result in increased preferential deamination activity on cytosine compared to 5mC include a single mutant with Y132P, and double mutants with a substitution mutation at Y130V and Y132H, or Y130W and Y132H. Specific examples of altered cytidine deaminases having increased cytidine deaminase activity and preferentially acts on cytosine compared to 5mC include SEQ ID NO: 7 or a sequence having at least 90%, at least 95%, at least 98%, at least 99% sequence identity to SEQ ID NO: 7 and comprising Y130L.

In one embodiment, the substitution mutation is at a position functionally equivalent to Y130 that results in 5hmC-defective deaminase activity (i.e., preferentially deaminates C and 5mC to U and T, respectively, and has significantly reduced deamination of 5hmC). In an exemplary aspect of this embodiment, the substitution mutation at a position functionally equivalent to Y130 is a mutation to an amino acid having a non-polar side chain or a hydrophobic side chain, such as tryptophan (W). Specific examples of altered cytidine deaminases having the ability to deaminate C and 5mC to U and T, respectively, but reduced ability to deaminate 5hmC, preferably no detectable ability to deaminate 5hmC include SEQ ID NO: 8 or a sequence having at least 90%, at least 95%, at least 98%, at least 99% sequence identity to SEQ ID NO: 8 and comprising Y130W.

In some embodiments, an altered cytidine deaminase includes a substitution mutation at a position functionally equivalent to tyrosine at position 130 (Y130) and/or tyrosine position 132 (Y132) in a member of the APOBEC3A subfamily. In some embodiments, such an altered cytidine deaminase demonstrates selective deamination for mC.

In some embodiments, an altered cytidine deaminase is an altered APOBEC3A cytidine deaminase, altered to include a substitution mutation at tyrosine at position 130 (Y130) and/or tyrosine position 132 (Y132). In some embodiments, such an altered APOBEC3A cytidine deaminase demonstrates selective deamination for mC.

In some embodiments, an altered cytidine deaminase is a double mutant of APOBEC3A, with substitution mutations Y130A/Y132H. In some embodiments, such an altered APOBEC3A cytidine deaminase demonstrates selective deamination for mC.

In some embodiments, an altered cytidine deaminase includes an altered cytidine deaminase having an amino acid of SEQ ID NO: 5, SEQ ID NO: 6, SEQ ID NO: 7, SEQ ID NO: 8, SEQ ID NO: 9, SEQ ID NO: 10, or SEQ ID NO: 11. In some embodiments, such an altered APOBEC3A cytidine deaminase demonstrates selective deamination for mC.

An altered cytidine deaminase described herein can include additional mutations. Typically, additional mutations do not unduly alter the activity of the altered cytidine deaminase. One or more additional mutations can be a conservative mutation.

An altered cytidine deaminase described herein can be a truncated protein. A truncated protein is a fragment of an altered cytidine deaminase of the present disclosure that retains the ability to deaminate 5mC to thymidine. A truncated altered cytidine deaminase can include a deletion of 1 to 13 amino acids on the N-terminal end of the protein, a deletion of 1 to 3 amino acids on the C-terminal end of the protein, or a combination thereof.

In some embodiments, an altered cytidine deaminase includes any of those described in International Patent Application No. PCT/US2023/017846 (“Altered Cytidine Deaminases and Methods of Use”), filed Apr. 7, 2023, which is hereby incorporated by reference in its entirety.

In general, methods for using a cytidine deaminase include contacting target nucleic acids, e.g., DNA or RNA, with the enzyme, under conditions suitable for conversion of modified cytidines, such as 5mC, to thymidine, or for conversion of unmodified cytidine to uracil. Because amplification of DNA does not preserve the modification status of cytidine (e.g., the methylation status of 5mC is not retained), use of a cytidine deaminase typically occurs before amplification of target DNA. Target nucleic acids can be contacted with cytidine deaminase at essentially any time. For instance, target nucleic acids can be contacted with cytidine deaminase after isolation of genomic or cell free DNA or mRNA, before or after fragmentation, or before or after tagmentation. The skilled person will recognize that target nucleic acids can be contacted with a cytidine deaminase after addition of a universal sequence and/or an adapter, provided the universal sequence and/or an adapter is not added by amplification.

Reaction conditions suitable for conversion of modified cytidines, such as 5mC, to thymidine by a cytidine deaminase include, but are not limited to, a substrate of target nucleic acid suspected of including at least one modified cytidine, with appropriate pH, temperature of the reaction, time of the reaction, and concentration of the cytidine deaminase and/or DNA or RNA substrate. It is expected that a cytidine deaminase can function in essentially any buffer. Examples of useful buffers include, but are not limited to, a citrate buffer, such as the citrate buffer available from Thermo Fisher Scientific (Cat. No. #005000); sodium acetate buffer, Bis Tris-Propane HCl; and Tris-HCl Tris. Examples of other buffers include, but are not limited to, Bicine, DIPSO, glycylglycine, HEPES, imidazole, malonate, MES, MOPS, PB, phosphate, PIPES, SPG, succinate, TAPS, TAPSO, trincine. Cytidine deaminases typically function at near-neutral pH, e.g., pH 7. In some embodiments a reducing agent such as dithiothreitol (DTT) can be present. In some embodiments a divalent cation is not included. A deamination reaction can occur at a temperature of about 25° C. to about 60° C., including but not limited to, at about 37° C., at about 45° C., at about 50° C., and at about 60° C.

Some cytidine deaminases preferentially deaminate a modified cytosine to thymidine at a faster rate than deamination of cytosine to uracil. Thus, in some embodiments the time of reaction can be used to allow the reaction to run to completion, to maximize the difference of deamination of modified cytosine versus deamination of cytosine. In some embodiments, the reaction can proceed for at least 15 minutes, at least 30 minutes, at least 45 minutes, at least 60 minutes, at least 90 minutes, at least 120 minutes, or at least 150 minutes, and for no greater than 15 minutes, no greater than 30 minutes, no greater than 45 minutes, no greater than 60 minutes, no greater than 90 minutes, no greater than 120 minutes, no greater than 150 minutes, or no greater than 180 minutes. In some embodiments, the reaction can run overnight.

In some embodiments, a deamination reaction can include a cytidine deaminase at a concentration from at least about 25 nanomolar (nM) to no greater than about 5 micromolar (μM). For instance, the concentration of the enzyme can be at least about 25 nM, at least about 0.5, at least about 1 μM, at least about 2 μM, at least about 3 μM, at least about 4 μM, or at least about 5 μM, and/or no greater than 5 μM, no greater than 4M, no greater than 3 μM, no greater than 2 μM, no greater than 1 μM, or 0.5 μM. In some embodiments, a deamination reaction can include about 1 ng to about 1 μg input nucleic acid. In some embodiments, a deamination reaction can include nucleic acids at a concentration of at least about 10 pM to at least about 200 nM.

### Amplification

At various stages in the methods described herein, DNA fragments may be amplified. It will be appreciated that any of the amplification methodologies described herein or generally known in the art may be used with universal or target-specific primers to amplify DNA fragments. Suitable methods for amplification include, but are not limited to, the polymerase chain reaction (PCR), strand displacement amplification (SDA), transcription mediated amplification (TMA) and nucleic acid sequence-based amplification (NASBA), as described in U.S. Pat. No. 8,003,354. The above amplification methods may be employed to amplify one or more nucleic acids of interest. For example, PCR, including multiplex PCR, SDA, TMA, NASBA and the like may be utilized to amplify DNA fragments. In some embodiments, primers directed specifically to the polynucleotide of interest are included in the amplification reaction.

As used herein, “amplify,” “amplifying” or “amplification reaction” and their derivatives, refer generally to any action or process whereby at least a portion of a nucleic acid molecule is replicated or copied into at least one additional nucleic acid molecule. The additional nucleic acid molecule optionally includes sequence that is substantially identical or substantially complementary to at least some portion of the target nucleic acid molecule. The target nucleic acid molecule can be single-stranded or double-stranded and the additional nucleic acid molecule can independently be single-stranded or double-stranded. Amplification optionally includes linear or exponential replication of a nucleic acid molecule. In some embodiments, such amplification can be performed using isothermal conditions; in other embodiments, such amplification can include thermocycling. In some embodiments, the amplification is a multiplex amplification that includes the simultaneous amplification of a plurality of target sequences in a single amplification reaction. In some embodiments, “amplification” includes amplification of at least some portion of DNA and RNA based nucleic acids alone, or in combination. The amplification reaction can include any of the amplification processes known to one of ordinary skill in the art.

As used herein, “amplification conditions” and its derivatives, generally refers to conditions suitable for amplifying one or more nucleic acid sequences. Such amplification can be linear or exponential. In some embodiments, the amplification conditions can include isothermal conditions or alternatively can include thermocyling conditions, or a combination of isothermal and thermocycling conditions. In some embodiments, the conditions suitable for amplifying one or more nucleic acid sequences include polymerase chain reaction (PCR) conditions. Typically, the amplification conditions refer to a reaction mixture that is sufficient to amplify nucleic acids such as one or more target sequences, or to amplify an amplified target sequence ligated to one or more adapters, e.g., an adapter-ligated amplified target sequence.

Generally, the amplification conditions include a catalyst for amplification or for nucleic acid synthesis, for example a polymerase; a primer that possesses some degree of complementarity to the nucleic acid to be amplified; and nucleotides, such as deoxyribonucleotide triphosphates (dNTPs) to promote extension of the primer once hybridized to the nucleic acid. The amplification conditions can require hybridization or annealing of a primer to a nucleic acid, extension of the primer and a denaturing step in which the extended primer is separated from the nucleic acid sequence undergoing amplification. Typically, but not necessarily, amplification conditions can include thermocycling; in some embodiments, amplification conditions include a plurality of cycles where the steps of annealing, extending, and separating are repeated. Typically, the amplification conditions include cations such as Mg++ or Mn++ and can also include various modifiers of ionic strength.

As used herein, the term “polymerase chain reaction” (PCR) refers to the method of K. B. Mullis as described in U.S. Pat. Nos. 4,683,195 and 4,683,202, which describes a method for increasing the concentration of a segment of a polynucleotide of interest in a mixture of genomic DNA without cloning or purification. This process for amplifying the polynucleotide of interest consists of introducing a large excess of two oligonucleotide primers to the DNA mixture containing the desired polynucleotide of interest, followed by a series of thermal cycling in the presence of a DNA polymerase. The two primers are complementary to their respective strands of the double-stranded polynucleotide of interest. The mixture is denatured at a higher temperature first and the primers are then annealed to complementary sequences within the polynucleotide of interest molecule. Following annealing, the primers are extended with a polymerase to form a new pair of complementary strands. The steps of denaturation, primer annealing, and polymerase extension can be repeated many times (referred to as thermocycling) to obtain a high concentration of an amplified segment of the desired polynucleotide of interest. The length of the amplified segment of the desired polynucleotide of interest (amplicon) is determined by the relative positions of the primers with respect to each other, and therefore, this length is a controllable parameter. By virtue of repeating the process, the method is referred to as the “polymerase chain reaction” (hereinafter “PCR”). Because the desired amplified segments of the polynucleotide of interest become the predominant nucleic acid sequences (in terms of concentration) in the mixture, they are said to be “PCR amplified.” In a modification to the method discussed above, the target nucleic acid molecules can be PCR amplified using a plurality of different primer pairs, in some cases, one or more primer pairs per target nucleic acid molecule of interest, thereby forming a multiplex PCR reaction.

In some embodiments, a uracil-tolerant polymerase may be employed for amplification. In some embodiments, a uracil-intolerant polymerase may be employed for amplification. In some embodiments, a uracil-intolerant polymerase may be employed for the first round of amplification, with subsequent rounds of amplification employing a uracil-tolerant polymerase.

### Sequencing

In some embodiments, DNA fragments obtained with amplification may be sequenced. Sequencing may be by any of a variety of known methodologies, including, but not limited to any of a variety high-throughput, next generation sequencing (NGS) platforms, including, but not limited to, sequencing by synthesis, sequencing by ligation, nanopore sequencing, Sanger sequencing, and the like. In some embodiments, sequencing is performed using the sequencing by synthesis methodologies commercialized by ILLUMINA® as described in U.S. Patent Application Publication No. 2007/0166705, U.S. Patent Application Publication No.

2006/0188901, U.S. Pat. No. 7,057,026, Beijing Genomics Institute (BG) as described in Carnevali et al., 2012, J Comput Biol; 9 (3): 279-92, or the ion semiconductor sequencing methodologies of ION TORRENT™ as described in US 2009/0026082 A1; US 2009/0127589 A1; US 2010/0137143 A1; or US 2010/0282617 A1, each of which is incorporated herein by reference.

Next Generation Sequencing (NGS) refers to sequencing methods that allow for massively parallel sequencing of clonally amplified molecules and of single nucleic acid molecules. Non-limiting examples of NGS include sequencing-by-synthesis using reversible dye terminators, and sequencing-by-ligation.

Preferred embodiments include sequencing-by-synthesis (SBS) techniques. SBS techniques generally involve the enzymatic extension of a nascent nucleic acid strand through the iterative addition of nucleotides against a template strand

In some embodiments, rather than sequencing, the readout may be obtained by the use of an array, using for example, procedures as described on the worldwide web illumina.com/techniques/microarrays/methylation-arrays.html.

### Kits

The present disclosure also provides kits for undertaking a method as described herein, for the reduction of false positive uracil residues due to deamination of unmethylated cytosines in an assay using a cytosine deaminase to deaminate methylated cytosines. In some embodiments, a kit may include at least one or more of a cytosine deaminase, primers, a uracil-tolerant polymerase, a uracil-intolerant polymerase, dNTPs, an uracil DNA glycosylase (UDG), and/or an endonuclease in a suitable packaging material in an amount sufficient for at least one reaction.

A kit may include one or more other components. Examples of other components include, for example, a positive control polynucleotide or a negative control polynucleotide.

Optionally, other reagents such as buffers and solutions are also included. Instructions for use of the packaged components are also typically included.

As used herein, the phrase “packaging material” refers to one or more physical structures used to house the contents of the kit. The packaging material is constructed by known methods, preferably to provide a sterile, contaminant-free environment. The packaging material has a label which indicates that the components can be used for the reducing uracil residues due to deamination of unmethylated cytosines in an assay using a cytosine deaminase to deaminate methylated cytosines. In addition, the packaging material contains instructions indicating how the materials within the kit are employed to practice a RUBY method as described herein. As used herein, the term “package” refers to a solid matrix or material such as glass, plastic, paper, foil, and the like, capable of holding within fixed limits the polypeptides. “Instructions for use” typically include a tangible expression describing the reagent concentration or at least one assay method parameter, such as the relative amounts of reagent and sample to be admixed, maintenance time periods for reagent/sample admixtures, temperature, buffer conditions, and the like.

### Exemplary Aspects

The invention is defined in the claims. However, below there is provided a non-exhaustive listing of non-limiting exemplary aspects. Any one or more of the features of these aspects may be combined with any one or more features of another example, embodiment, or aspect described herein. Exemplary Embodiments of the present invention include, but are not limited to, the following.

Aspect 1 is a method of preventing false positive detection of 5-methylcytosine (5mC) and/or 5-hydroxymethylcytosine (5hmC) due to deamination of unmethylated cytosines in an assay using a cytosine deaminase to selectively deaminate methylated cytosines, the method comprising:
providing a sample comprising DNA library fragments in which a cytosine deaminase has deaminated methylated cytosines, wherein the DNA library fragments comprise 5′ end and 3′ end library adapters; and
subjecting the sample to at least one round of second strand synthesis by contacting the sample comprising DNA library fragments with an uracil-intolerant polymerase, dNTPs, and primers complementary to the 5′ end and 3′ end library adapters under conditions to provide for second strand synthesis resulting in double stranded DNA uracil-free library fragments.

Aspect 2 is the method of aspect 1, further comprising subjecting the sample comprising double stranded DNA uracil-free library fragments to polymerases chain reaction (PCR) amplification with either a uracil-tolerant polymerase or a uracil-intolerant polymerase.

Aspect 3 is a method of preventing false positive detection of 5-methylcytosine (5mC) and/or 5-hydroxymethylcytosine (5hmC) due to deamination of unmethylated cytosines in an assay using a cytosine deaminase to selectively deaminate methylated cytosines, the method comprising:
providing a sample comprising library DNA fragments in which a cytosine deaminase has deaminated methylated cytosines, wherein the DNA library fragments comprise 5′ end and 3′ end library adapters; and
contacting the sample comprising DNA library fragments with an uracil DNA glycosylase (UDG) and an endonuclease resulting in DNA library fragments cleaved at uracil residues; and
subjecting the sample comprising DNA library fragments cleaved at uracil residues to polymerase chain reaction (PCR) amplification by contacting the sample comprising DNA library fragments cleaved at uracil residues with a polymerase, dNTPs, and primers complementary to the 5′ end and 3′ end library adapters under conditions to provide for second strand synthesis resulting in double stranded DNA uracil-free library fragments.

Aspect 4 is the method of aspect 3, wherein the polymerase comprises an uracil-intolerant polymerase.

Aspect 5 is the method of any one of aspects 1 to 4, wherein the uracil-intolerant polymerase comprises KAPA HiFi, Ultra II Q5, or Phusion HiFi.

Aspect 6 is a method of preventing false positive detection of 5-methylcytosine (5mC) and/or 5-hydroxymethylcytosine (5hmC) due to deamination of unmethylated cytosines in an assay using a cytosine deaminase to selectively deaminate methylated cytosines, the method comprising:
providing a sample comprising original DNA library fragments in which a cytosine deaminase has deaminated methylated cytosines, wherein the original DNA library fragments comprise 5′ end and 3′ end library adapters;
subjecting the sample to second strand synthesis by contacting the sample comprising original DNA library fragments with an uracil-intolerant polymerase, dNTPs, and primers complementary to the 5′ end and 3′ end library adapters under conditions to provide for second strand synthesis resulting in DNA library fragments without uracil residues in the synthesized second strand;
contacting the sample comprising DNA library fragments without uracil residues in the synthesized second strand with an endonuclease to digest the original DNA library fragments resulting in single stranded synthesized second strands; and
subjecting the sample comprising single stranded synthesized second strands to polymerase chain reaction (PCR) amplification by contacting the sample comprising single stranded synthesized second strands with a polymerase, dNTPs, and primers complementary to the 5′ end and 3′ end library adapters under conditions to provide for second strand synthesis resulting in double stranded DNA uracil-free library fragments.

Aspect 7 is the method of aspect 6 wherein subjecting the sample comprising single stranded synthesized second strands to PCR amplification comprises contacting the sample comprising single stranded synthesized second strands with a uracil tolerant polymerase.

Aspect 8 is the method of any one of aspects 1 to 7, wherein the DNA library fragments comprise single stranded DNA library fragments.

Aspect 9 is the method of any one of aspects 1 to 7, wherein the DNA library fragments comprise double stranded DNA library fragments.

Aspect 10 is the method of any one of aspects 1 to 9, wherein the cytosine deaminase comprises an altered cytosine deaminase.

Aspect 11 is a method of aspect 10, wherein the altered cytosine deaminase is a member of the AID subfamily, the APOBEC1 subfamily, the APOBEC2 subfamily, the APOBEC3A subfamily, the APOBEC3B subfamily, the APOBEC3C subfamily, the APOBEC3D subfamily, the APOBEC3F subfamily, the APOBEC3G subfamily, the APOBEC3G subfamily, the APOBEC3H subfamily, or the APOBEC4 subfamily, or an alteration thereof.

Aspect 12 is the method of aspect 10, wherein the altered cytosine deaminase comprises an altered APOBEC3A.

Aspect 13 is the method of any one of aspects 10 to 12, wherein the altered cytidine deaminase comprises an amino acid substitution mutation at a position functionally equivalent to (Tyr/Phe) 130 in a wild-type APOBEC3A protein and/or an amino acid substitution mutation at a position functionally equivalent to Tyr132 in a wild-type APOBEC3A protein.

Aspect 14 is the method of any one of aspects 10 to 13, wherein the altered cytidine deaminase comprises amino acid substitution mutations at positions functionally equivalent to (Tyr/Phe) 130 and Tyr132 in a wild-type APOBEC3A protein.

Aspect 15 is the method of any one of aspect 10 to 14, wherein the altered cytidine deaminase comprises an amino acid substitution mutation at a position functionally equivalent to (Tyr/Phe) 130 in a wild-type APOBEC3A protein, wherein the substitution mutation is (Tyr/Phe) 130Trp.

Aspect 16 is the method of aspect 14 or 15, wherein the (Tyr/Phe) 130 is Tyr130, and the wild-type APOBEC3A protein is SEQ ID NO: 12.

Aspect 17 is the method of any one of aspect 10 to 16, wherein the substitution mutation at the position functionally equivalent to Tyr130 comprises a mutation to alanine, glycine, phenylalanine, histidine, glutamine, methionine, asparagine, lysine, valine, aspartic acid, glutamic acid, serine, cysteine, proline, arginine, or threonine.

Aspect 18 is the method of any one of aspect 13 to 17, wherein the substitution mutation at the position functionally equivalent to Tyr130 comprises a mutation to Ala, Val, or Trp.

Aspect 19 is the method of any one of aspects 13 or 18, wherein the substitution mutation at the position functionally equivalent to Tyr132 comprises a mutation to His, Arg, Gln, or Lys.

Aspect 20 is the method of any one of aspects 10 to 19, wherein the altered cytidine deaminase converts 5-methyl cytosine (5mC) to thymidine (T) by deamination at a greater rate than conversion of cytosine (C) to uracil (U) by deamination.

Aspect 21 is the method of aspect 20, wherein the rate is at least 100-fold greater.

Aspect 22 is the method of any one of aspects 10 to 21, wherein the altered cytidine deaminase converts cytosine (C) to uracil (U) by deamination and 5-methyl cytosine (5mC) to thymidine (T) by deamination at a greater rate than conversion of 5-hydroxymethyl cytosine (5hmC) to 5-hydroxymethyl uracil (5hmU) by deamination.

Aspect 23 is the method of aspect 22, wherein conversion of 5hmC to 5hmU by deamination is undetectable.

Aspect 24 is the method of any one of aspects 10 to 23, wherein the altered cytidine deaminase comprises a ZDD motif H-[P/A/V]-E-X[23-28]-P—C-X_{[2-4}]-C(SEQ ID NO: 1).

Aspect 25 is the method of any one of aspects 10 to 24, wherein the altered cytidine deaminase is a member of the APOBEC3A subfamily and comprises a ZDD motif HXEX₂₄SW(S/T)PCX_{[2-4]}CX₆FX₈LX5R(L/I)YX_{[8-11]}LX2LX_{[10]}M (SEQ ID NO: 2), wherein the amino acid substitution mutation at the position functionally equivalent to (Tyr/Phe) 130 of the wild-type APOBEC3A protein is the Tyr (Y) amino acid of the ZDD motif.

Aspect 26 is the method of any one of aspects 10 to 25, wherein the altered cytidine deaminase is a member of the APOBEC3A subfamily and comprises X_{[16-26]}-GRXXTXLCYXV-X₁₅-GXXXN-X₁₂-HAEXXF-X₁₄-YXXTWXXSWSPC-X_{[2-4}]-CA-X₅-FL-X₇-LXIXXXR(L/I)Y-X₈-GLXXLXXXG-X₅-M-X₄-FXXCWXXFV-X₆-FXPW-X₁₃-LXXI-X_{[2-6]} (SEQ ID NO: 3).

Aspect 27 is the method of any one of aspects 10 to 26, wherein the altered cytidine deaminase is a member of the APOBEC3A family and comprises SEQ ID NO: 5, SEQ ID NO: 6, SEQ ID NO: 7, SEQ ID NO: 8, SEQ ID NO: 9, SEQ ID NO: 10, or SEQ ID NO: 11.

Aspect 28 is the method of any one of aspects 1 to 27, wherein the DNA library fragments are about 100 bp to about 300 bp in length.

Aspect 29 is the method of any one of aspects 1 to 28 further comprising sequencing the double stranded DNA uracil-free library fragments.

Aspect 30 is the method of any one of aspects 1 to 29 further comprising processing the double stranded DNA uracil-free library fragments to produce a sequencing library.

Aspect 31 is the method of aspect 30, further comprising sequencing the sequencing library.

Aspect 32 is a kit comprising a cytosine deaminase and an uracil intolerant polymerase. Aspect 33 is a kit comprising a cytosine deaminase, an uracil DNA glycosylase (UDG), and an AP endonuclease.

Aspect 34 is the kit of aspect 33, wherein the AP endonuclease comprises Endonuclease IV and/or AP Endonuclease I.

Aspect 35 is a method of removing DNA fragments comprising uracil residues from a sample, the method comprising:
providing a sample comprising DNA fragments, wherein the DNA fragments comprise 5′ end and 3′ end library adapters; and
subjecting the sample to polymerase chain reaction (PCR) amplification by contacting the sample comprising DNA fragments with an uracil-intolerant polymerase, dNTPs, and primers complementary to the 5′ end and 3′ end library adapters under conditions to provide for second strand synthesis resulting in double stranded DNA uracil-free fragments.

Aspect 36 is the method of aspect 35, further comprising subjecting the sample comprising double stranded DNA uracil-free library fragments to polymerases chain reaction (PCR) amplification with either a uracil-tolerant polymerase or a uracil-intolerant polymerase.

Aspect 37 is a method of removing DNA fragments comprising uracil residues from a sample, the method comprising:
providing a sample comprising DNA fragments, wherein the DNA fragments comprise 5′ end and 3′ end library adapters; and
contacting the sample comprising DNA fragments with an uracil DNA glycosylase (UDG) and an endonuclease resulting in DNA fragments cleaved at uracil residues; and
subjecting the sample comprising DNA fragments cleaved at uracil residues to polymerase chain reaction (PCR) amplification by contacting the sample comprising DNA fragments cleaved at uracil residues with a polymerase, dNTPs, and primers complementary to the 5′ end and 3′ end library adapters under conditions to provide for second strand synthesis resulting in double stranded DNA uracil-free fragments.

Aspect 38 is a method of aspect 37, wherein the polymerase comprises an uracil intolerant polymerase.

Aspect 39 is the method of any one of aspects 35 to 38, wherein the uracil-intolerant polymerase comprises KAPA HiFi, Ultra II Q5, or Phusion HiFi.

Aspect 40 is a method of removing DNA fragments comprising uracil residues from a sample, the method comprising:
providing a sample comprising original DNA fragments, wherein the original DNA fragments comprise 5′ end and 3′ end library adapters;
subjecting the sample to second strand synthesis by contacting the sample comprising original DNA fragments with an uracil-intolerant polymerase, dNTPs, and primers complementary to the 5′ end and 3′ end library adapters under conditions to provide for second strand synthesis resulting in DNA fragments without uracil residues in the synthesized second strand;
contacting the sample comprising DNA fragments without uracil residues in the synthesized second strand with an endonuclease to digest the original DNA fragments resulting in single stranded synthesized second strands; and
subjecting the sample comprising single stranded synthesized second strands to polymerase chain reaction (PCR) amplification by contacting the sample comprising single stranded synthesized second strands with a polymerase, dNTPs, and primers complementary to the 5′ end and 3′ end library adapters under conditions to provide for second strand synthesis resulting in double stranded DNA uracil-free fragments.

Aspect 41 is the method of aspect 40 wherein subjecting the sample comprising single stranded synthesized second strands to PCR amplification comprises contacting the sample comprising single stranded synthesized second strands with a uracil tolerant polymerase.

Aspect 42 is the method of any one of aspects 35 to 41, wherein the DNA library fragments comprise single stranded DNA library fragments.

Aspect 43 is the method of any one of aspects 35 to 41, wherein the DNA library fragments comprise double stranded DNA library fragments.

Aspect 44 is the method of any one of aspects 35 to 43, wherein the DNA library fragments are about 100 bp to about 300 bp in length.

Aspect 45 is the method of any one of aspects 35 to 44 further comprising sequencing the double stranded DNA uracil-free library fragments.

Aspect 46 is the method of any one of aspects 35 to 45 further comprising processing the double stranded DNA uracil-free library fragments to produce a sequencing library. Aspect 47 is the method of aspect 46, further comprising sequencing the sequencing library.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Uracil Intolerant Polymerases and USER Both Reduce Methylation False Positive FP Rates

NA12878 genomic DNA was combined with fully unmethylated lambda control DNA and enzymatically CpG methylated pUC19 control DNA and mechanically sheared to give fragments of approximately ˜200 bp. This sheared DNA (50 ng) was then subjected to end-repair, A-tailing, and adapter ligation according to standard library preparation procedures. The adapter ligated DNA was denatured via incubation in 0.02 N sodium hydroxide at 50° C. for 10 minutes. Subsequently, this ssDNA sample was enzymatically deaminated in 50 mM Bis-Tris (pH 6.5), 1 mM DTT, 0.2 mg/mL BSA with an engineered cytidine deaminase (750 nM) for 3 hours at 37° C. After SPRI-based purification, some of these libraries were subjected to additional treatment by 1 unit of USER (NEB) in rCutSmart Buffer (NEB) for 30 minutes at 37° C., followed by an additional SPRI-purification. The libraries were then PCR amplified using unique-dual indexing primers, using either a uracil tolerant polymerase (KAPA HiFi Uracil+, Roche) or a uracil intolerant polymerase (KAPA HiFi, Roche) using 12 cycles of PCR. Libraries were then sequenced on a NextSeq550 and down sampled to 5 million paired-end reads per sample, and analysis was performed with DRAGEN Methylation Pipeline.

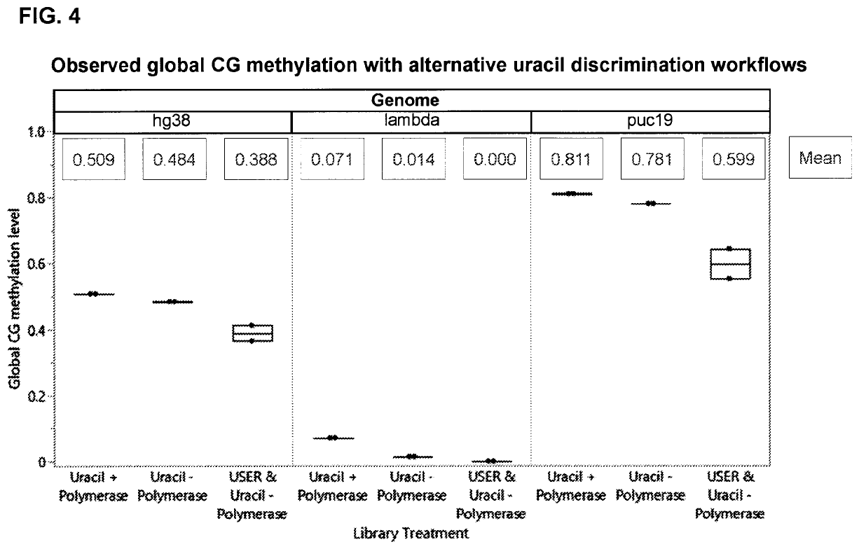

FIG. 4 shows global methylation levels for different genomes when the assay was performed with a uracil tolerant (+) polymerase, a uracil intolerant (−) polymerase, or both USER treatment and a uracil (−) polymerase. The libraries that were amplified with the uracil intolerant polymerase had strongly reduced false positive methylation, as evidenced by the lambda CG methylation level decreasing from 0.071 to 0.014 (FIG. 4). A small decrease in mC detection was also observed, as evidenced by the lower pUC19 methylation (0.781 compared to 0.811).

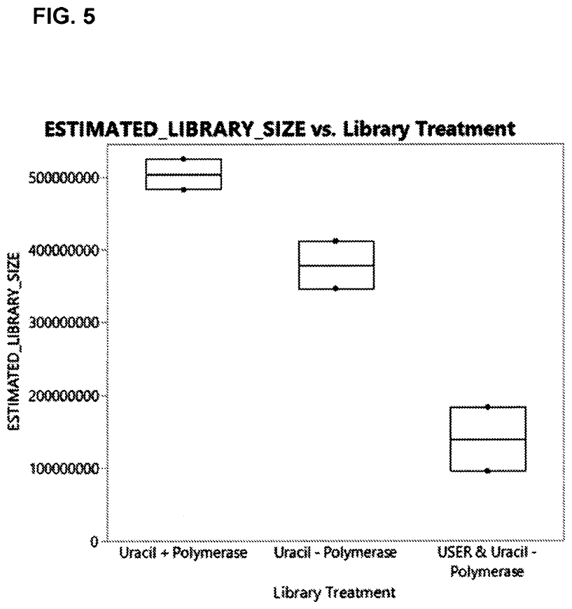

The libraries that were treated with USER and subsequently amplified with a uracil intolerant polymerase had even higher specificity, with a greatly reduced methylation level for the unmethylated lambda genome (0.00022). A reduction in overall mC detection was also observed in these samples. Using Picard Estimate Library Complexity Metrics, the library complexity for these differentially treated libraries was also compared. The estimated library size, a measure of library complexity, was found to be slightly lower for those libraries amplified with the uracil intolerant polymerase, and further reduced for libraries treated with USER and a uracil intolerant polymerase (FIG. 5).

This example demonstrates that uracil discrimination reduces false positive methylation reporting.

### Example 2

### Assessment of Alternative Uracil-Intolerant Polymerases

Alternative polymerase pairs were evaluated in this workflow to characterize the level of uracil discrimination and overall performance. NA12878 genomic DNA was combined with fully unmethylated lambda control DNA and enzymatically CpG methylated pUC19 control DNA and mechanically sheared to give fragments of approximately ˜300 bp. This sheared DNA (50 ng) was then subjected to end-repair, A-tailing, and adapter ligation according to standard library preparation procedures. The adapter ligated DNA was denatured via incubation in 0.02 N sodium hydroxide at 50° C. for 10 minutes. Subsequently, this ssDNA sample was enzymatically deaminated in 50 mM Bis-Tris (pH 6.5), 1 mM DTT, 0.2 mg/mL BSA with the cytidine deaminase (135 nM) for 30 minutes at 37° C. After SPRI-based purification, the libraries were split into two separate reactions, where they were amplified by either a U-tolerant polymerase or a U-intolerant polymerase of the same family using 12 cycles of PCR. The polymerase families tested are listed in Table 2.

TABLE 2
Uracil tolerant and uracil intolerant PCR polymerase
pairs evaluated in the methylation assay.

| | Uracil tolerant | Uracil intolerant |
|---|---|---|
| Kapa (Roche) | KapaU | Kapa HiFi |
| Q5 (NEB) | Q5U | Ultra II Q5 |
| Phusion | Phusion U | Phusion HiFi |
| (Fisher Scientific) | | |

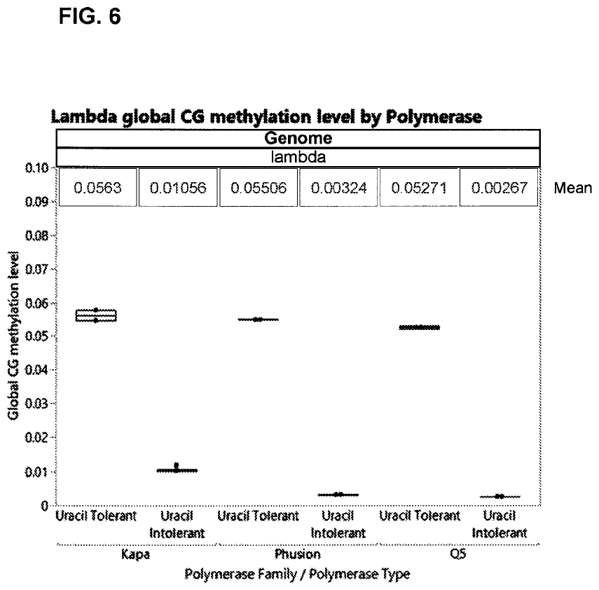

Libraries were then sequenced on a NextSeq 550 and down sampled to 5 million paired-end reads per sample, and analysis was performed with DRAGEN Methylation Pipeline. Analysis showed that all uracil intolerant polymerases tested were capable of reducing the false positive methylation rate reported on the lambda genome, with Q5 and Phusion polymerases showing a greater reduction in the false positive rate (FIG. 6).

### Example 3

### Methylation Assay with U-Intolerant Polymerase on Human Genomic DNA

In order to evaluate the performance of U-intolerant polymerases in the methylation assay on human genomic DNA, NA12878 genomic DNA was combined with fully unmethylated lambda control DNA and enzymatically CpG methylated pUC19 control DNA and mechanically sheared to give fragments of approximately ˜300 bp. This sheared DNA (50 ng) was then subjected to end-repair, A-tailing, and adapter ligation according to standard Illumina library preparation procedures. The adapter ligated DNA was denatured via incubation in 0.02 N sodium hydroxide at 50° C. for 10 minutes. Subsequently, ssDNA samples were enzymatically deaminated in 50 mM Bis-Tris (pH 6.5), 1 mM DTT, 0.2 mg/mL BSA, 5 μg/mL RNAse A, 1M betaine with the cytidine deaminase (200 nM) for 30 minutes at 37° C. The libraries were then PCR amplified using unique-dual indexing primers, using either a uracil tolerant polymerase (Q5U, New England Biolabs) or a uracil intolerant polymerase (Q5 HiFi, New England Biolabs) using 12 cycles of PCR. Samples were sequenced on a NovaSeq6000.

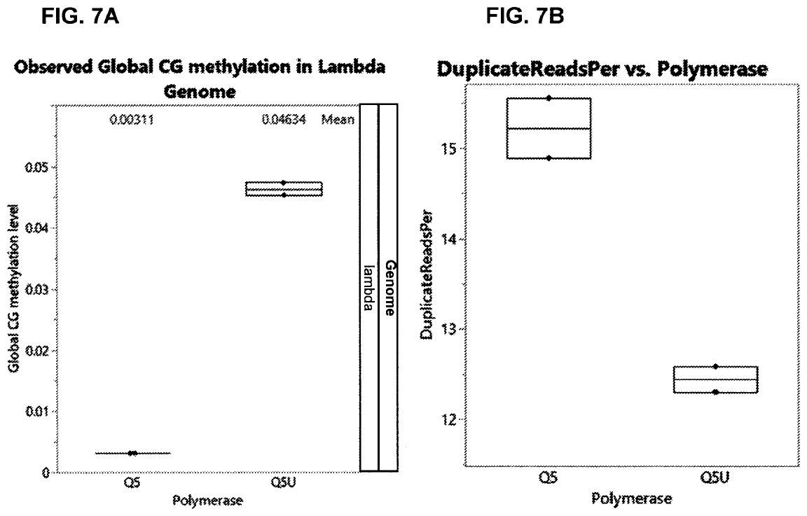

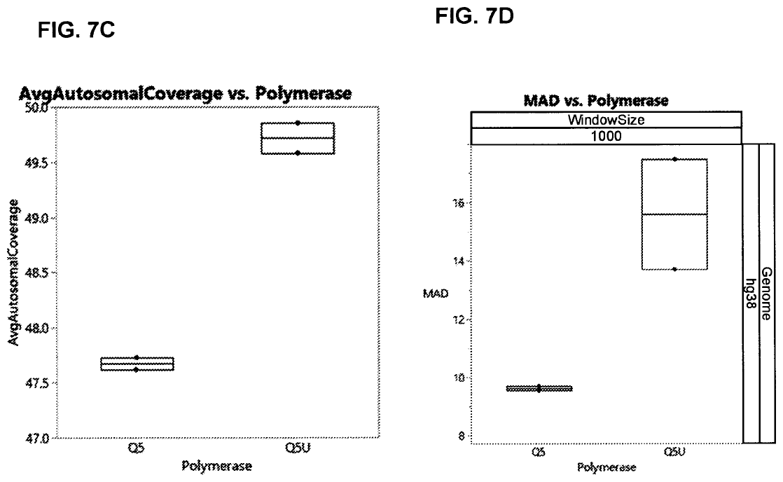

Analysis of the sequencing data showed that the use of Q5 HiFi led to a small increase in duplicate reads, and a corresponding small decrease in autosomal coverage relative to Q5U (FIGS. 7A-7D). FIG. 7A shows false positive rate observed in the lambda genome in this experiment. FIG. 7B presents duplicate reads. FIG. 7C presents average autosomal coverage over the human genome. FIG. 7D presents median average deviation (MAD) of coverage. Uniformity of coverage, as measured using a median average deviation (MAD) metric was slightly improved by the use of Q5 HiFi (indicated by the lower MAD value). Accordingly, despite removal of some reads through the use of a U-intolerant polymerase, the libraries generated maintain good quality with observed differences having little practical impacts on performance.

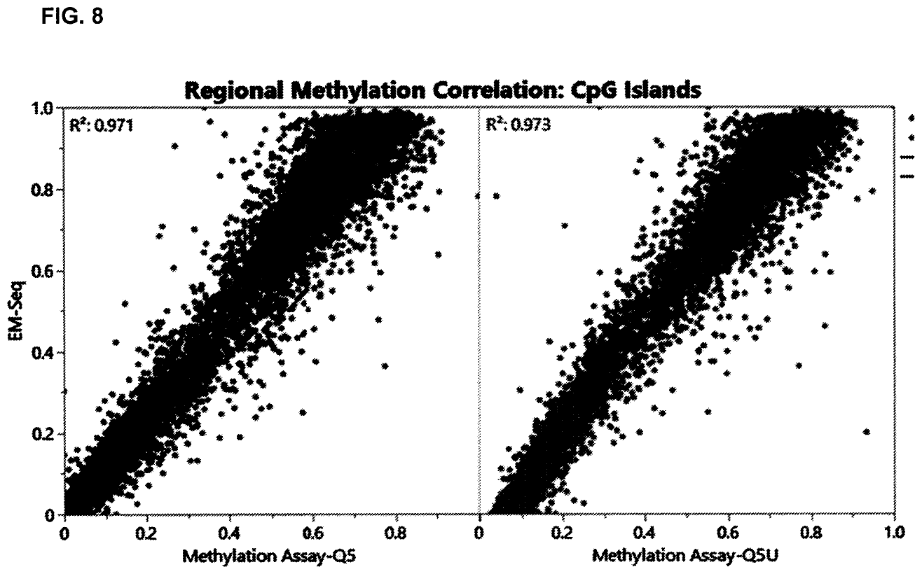

In order to assess the methylation performance on the human genome, regional methylation values for CpG islands across the human genome were calculated using methylpy. An EM-seq dataset on NA12878 was also analyzed for comparison. The per-region methylation values were plotted against the per-region methylation values for EM-seq (FIG. 8). This analysis showed good correlation of the methylation values generated with Q5 Polymerase to those reported with EM-Seq, with an R-squared value of 0.971. For comparison, the R-squared for the data generated with Q5U Polymerase was 0.973.

This example demonstrates that uracil discrimination produces good quality sequencing and methylation reporting.

### Example 4

### Differential Methylation Analysis

A common application for methylation NGS assays is the identification of differentially methylated regions (DMRs), which are genomic regions with different methylation levels across multiple DNA samples, which can be used to profile epigenetic changes in cancer and differences across tissue types (Chen et al., 2016, Briefings in Functional Genomics; 15 (6): 485-90). In order to characterize the performance of the APOBEC methylation assay using a uracil intolerant polymerase, two different samples of human genomic DNA were used: NA12878 DNA and HeLa DNA. Both of these samples were supplemented with small amounts of control DNA samples (unmethylated lambda DNA and enzymatically CpG methylated pUC19 DNA) and sheared to an average fragment size of approximately 300 bp. This sheared DNA (100 ng) was then subjected to end-repair, A-tailing, and adapter ligation according to standard library preparation procedures. The adapter ligated DNA was denatured via incubation in 0.02 N sodium hydroxide at 50° C. for 10 minutes. Subsequently, ssDNA samples were enzymatically deaminated in 50 mM Bis-Tris (pH 6.5), 1 mM DTT, 0.2 mg/mL BSA with APOBEC-Y130AY132H (750 nM) for 30 minutes at 37° C. The libraries were then PCR amplified using unique-dual indexing primers, using either a uracil tolerant polymerase (KAPA HiFi Uracil+, Roche) or a uracil intolerant polymerase (KAPA HiFi, Roche) using 10 cycles of PCR. Comparative libraries were also treated with the EM-Seq conversion kit and amplified with Q5U PCR according to the manufacturer's recommendations. Samples were sequenced on a NovaSeq6000 and down sampled to 680 million paired-end reads for analysis according to the same procedures described above.

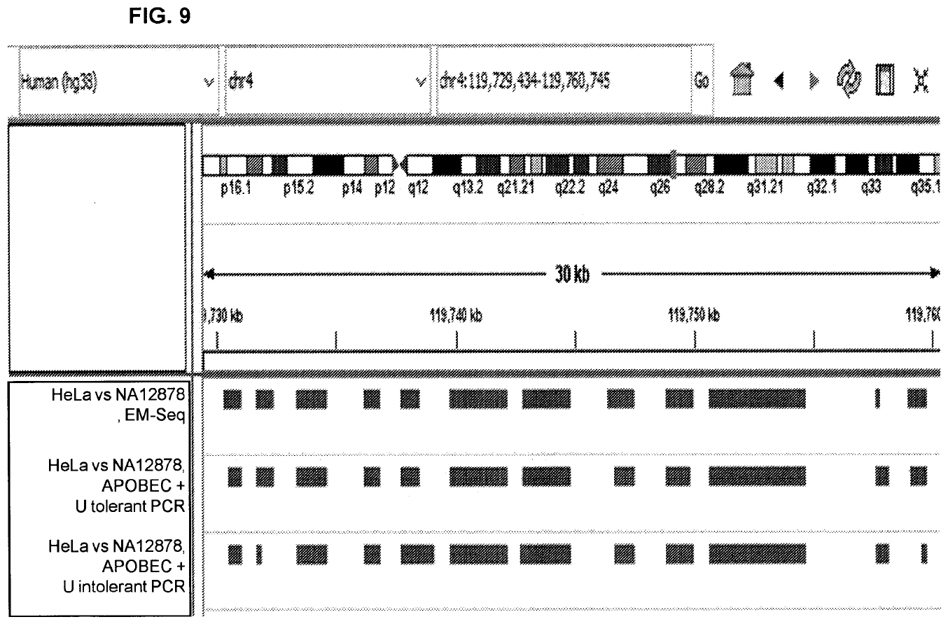

For differential methylation analysis, HOME, a program for identifying DMRs, was used (Srivastava et al., 2019, BMC Bioinformatics; 20 (1): 253). Comparisons of the methylation between NA12878 and HeLa samples were performed for each methylation assay. To evaluate the performance of the APOBEC-based assays against the comparative EM-seq method, a Jaccard statistic was calculated using bedtools jaccard, with the default settings. The Jaccard statistic evaluates the similarity between two sets of genomic intervals and ranges between 0 and 1, where 0 represents no overlap and 1 represents complete overlap (worldwide web at bedtools.readthedocs.io/en/latest/content/tools/jaccard.html). The Jaccard indexes for both the U-intolerant and U-tolerant polymerases were similar, suggesting that the U-intolerant polymerase does not interfere with the assay (Table 3). Visualization of the identified regions also shows high levels of similarity of the described methods with EM-Seq (FIG. 9).

TABLE 3
Jaccard index for comparison of the DMRs identified between
NA12878 and HeLa, using EM-Seq data for comparison.

| Workflow | Jaccard Index |
|---|---|
| APOBEC assay + Uracil tolerant polymerase | 0.726 |
| APOBEC assay + Uracil intolerant polymerase | 0.736 |

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

SEQUENCE INFORMATION

SEQ ID NO: 1
zinc-binding motif H-[P/A/V]-E-X[23-28]-P-C-X[2-4]-C
SEQ ID NO: 2
ZDD motif: HXEX₂₄SW(S/T)PCX_{[2-4]}CX₆FX₈LX₅R(L/I)YX_{[8-11]}LX₂LX_{[10]}M
SEQ ID NO: 3
altered cytidine deaminase includes the amino acids of a member of the APOBEC3A subfamily:
X_{[16-26]}-GRXXTXLCYXV-X₁₅-GXXXN-X₁₂-HAEXXF-X₁₄-YXXTWXXSWSPC- X_{[2-4]}-CA-
X₅-FL-X₇-LXIXXXR(L/I)Y-X₈-GLXXLXXXG-X₅-M-X₄-FXXCWXXFV-X₆-FXPW-X₁₃-
LXXI- X_{[2-6]}
SEQ ID NO: 4
altered cytidine deaminase includes the amino acids of a member of the APOBEC3A subfamily:
X₂₆-GRXXTXLCYXV-X₁₅-G-X₁₆-HAEXXF-X₁₄-YXXTWXXSWSPC-X₄-CA-X₅-FL-X₇-
LXIFXXR(L/I)Y-X₈-GLXXLXXXG-X₅-M-X₄-FXXCWXXFV-X₆-FXPW-X₁₃-LXXI-X₆
SEQ ID NO: 5
Altered cytosine deaminase (SG1) - synthetic construct:
MEASPASGPRHLMDPHIFTSNFNNGIGRHKTYLCYEVERLDNGTSVKMDQHRGFLHNQ
AKNLLCGFYGRHAELRFLDLVPSLQLDPAQIYRVTWFISWSPCFSWGCAGEVRAFLQEN
THVRLRIFAARIADYDPLYKEALQMLRDAGAQVSIMTYDEFKHCWDTFVDHQGCPFQP
WDGLDEHSQALSGRLRAILQNQGN
SEQ ID NO: 6
Altered cytosine deaminase (SG2) - synthetic construct:
MEASPASGPRHLMDPHIFTSNFNNGIGRHKTYLCYEVERLDNGTSVKMDQHRGFLHNQ
AKNLLCGFYGRHAELRFLDLVPSLQLDPAQIYRVTWFISWSPCFSWGCAGEVRAFLQEN
THVRLRIFAARIADHDPLYKEALQMLRDAGAQVSIMTYDEFKHCWDTFVDHQGCPFQP
WDGLDEHSQALSGRLRAILQNQGN
SEQ ID NO: 7
Altered cytosine deaminase- synthetic construct
APOBECC3A with (Y130L)
MEASPASGPRHLMDPHIFTSNFNNGIGRHKTYLCYEVERLDNGTSVKMDQHRGFLHNQ
AKNLLCGFYGRHAELRFLDLVPSLQLDPAQIYRVTWFISWSPCFSWGCAGEVRAFLQEN
THVRLRIFAARILDYDPLYKEALQMLRDAGAQVSIMTYDEFKHCWDTFVDHQGCPFQP
WDGLDEHSQALSGRLRAILQNQGN
SEQ ID NO: 8
Altered cytosine deaminase - synthetic construct
APOBEC3A with (Y130W)
MEASPASGPRHLMDPHIFTSNFNNGIGRHKTYLCYEVERLDNGTSVKMDQHRGFLHNQ
AKNLLCGFYGRHAELRFLDLVPSLQLDPAQIYRVTWFISWSPCFSWGCAGEVRAFLQEN
THVRLRIFAARIWDYDPLYKEALQMLRDAGAQVSIMTYDEFKHCWDTFVDHQGCPFQP
WDGLDEHSQALSGRLRAILQNQGN
SEQ ID NO: 9
Altered cytosine deaminase (SG1 with Y130X)
MEASPASGPRHLMDPHIFTSNFNNGIGRHKTYLCYEVERLDNGTSVKMDQHRGFLHNQ
AKNLLCGFYGRHAELRFLDLVPSLQLDPAQIYRVTWFISWSPCFSWGCAGEVRAFLQEN
THVRLRIFAARIXDYDPLYKEALQMLRDAGAQVSIMTYDEFKHCWDTFVDHQGCPFQP
WDGLDEHSQALSGRLRAILQNQGN (wherein X can be A, G, F, H, Q, M, N, K, V, D, E, S,
C, P, or T, preferably A)
SEQ ID NO: 10
Altered cytosine deaminase - synthetic construct
X26-GRXXTXLCYXV-X15-G-X16-HAEXXF-X14-YXXTWXXSWSPC-X4-CA-X5-FL-X7-
LXIFXXR(L/I)Z-X8-GLXXLXXXG-X5-M-X4-FXXCWXXFV-X6-FXPW-X13-LXXI-X6
(wherein Z is A, G, F, H, Q, M, N, K, V, D, E, S, C, P, or T, and the number after an X refers to
the number of amino acids present)
SEQ ID NO: 11
Altered cytosine deaminase - synthetic construct
MEASPASGPRHLMDPHIFTSNFNNGIGRHKTYLCYEVERLDNGTSVKMDQHRGFLHNQ
AKNLLCGFYGRHAELRFLDLVPSLQLDPAQIYRVTWFISWSPCFSWGCAGEVRAFLQEN
THVRLRIFAARIXDZDPLYKEALQMLRDAGAQVSIMTYDEFKHCWDTFVDHQGCPFQP
WDGLDEHSQALSGRLRAILQNQGN, (wherein X can be A, L, or W, preferably A; and Z is
selected from R, H, L, or Q, preferably H).
SEQ ID NO: 12
Wild Type human APOBEC3A protein (UniProt: P31941)
MEASPASGPRHLMDPHIFTSNFNNGIGRHKTYLCYEVERLDNGTSVKMDQHRGFLHNQ
AKNLLCGFYGRHAELRFLDLVPSLQLDPAQIYRVTWFISWSPCFSWGCAGEVRAFLQEN
THVRLRIFAARIYDYDPLYKEALQMLRDAGAQVSIMTYDEFKHCWDTFVDHQGCPFQP
WDGLDEHSQALSGRLRAILQNQGN

1. A method of preventing false positive detection of 5-methylcytosine (5mC) and/or 5-hydroxymethylcytosine (5hmC) due to deamination of unmethylated cytosines in an assay using a cytosine deaminase to selectively deaminate methylated cytosines, the method comprising:
providing a sample comprising DNA library fragments in which a cytosine deaminase has deaminated methylated cytosines, wherein the DNA library fragments comprise 5′ end and 3′ end library adapters; and
subjecting the sample to at least one round of second strand synthesis by contacting the sample comprising DNA library fragments with an uracil-intolerant polymerase, dNTPs, and primers complementary to the 5′ end and 3′ end library adapters under conditions to provide for second strand synthesis resulting in double stranded DNA uracil-free library fragments.

2. The method of claim 1, further comprising subjecting the sample comprising double stranded DNA uracil-free library fragments to polymerases chain reaction (PCR) amplification with either a uracil-tolerant polymerase or a uracil-intolerant polymerase.

3. A method of preventing false positive detection of 5-methylcytosine (5mC) and/or 5-hydroxymethylcytosine (5hmC) due to deamination of unmethylated cytosines in an assay using a cytosine deaminase to selectively deaminate methylated cytosines, the method comprising:
providing a sample comprising library DNA fragments in which a cytosine deaminase has deaminated methylated cytosines, wherein the DNA library fragments comprise 5′ end and 3′ end library adapters; and
contacting the sample comprising DNA library fragments with an uracil DNA glycosylase (UDG) and an endonuclease resulting in DNA library fragments cleaved at uracil residues; and
subjecting the sample comprising DNA library fragments cleaved at uracil residues to polymerase chain reaction (PCR) amplification by contacting the sample comprising DNA library fragments cleaved at uracil residues with a polymerase, dNTPs, and primers complementary to the 5′ end and 3′ end library adapters under conditions to provide for second strand synthesis resulting in double stranded DNA uracil-free library fragments.

4. The method of claim 3, wherein the polymerase comprises an uracil-intolerant polymerase.

5. The method of claim 1, wherein the uracil-intolerant polymerase comprises KAPA HiFi, Ultra II Q5, or Phusion HiFi.

6. A method of preventing false positive detection of 5-methylcytosine (5mC) and/or 5-hydroxymethylcytosine (5hmC) due to deamination of unmethylated cytosines in an assay using a cytosine deaminase to selectively deaminate methylated cytosines, the method comprising:
providing a sample comprising original DNA library fragments in which a cytosine deaminase has deaminated methylated cytosines, wherein the original DNA library fragments comprise 5′ end and 3′ end library adapters;
subjecting the sample to second strand synthesis by contacting the sample comprising original DNA library fragments with an uracil-intolerant polymerase, dNTPs, and primers complementary to the 5′ end and 3′ end library adapters under conditions to provide for second strand synthesis resulting in DNA library fragments without uracil residues in the synthesized second strand;
contacting the sample comprising DNA library fragments without uracil residues in the synthesized second strand with an endonuclease to digest the original DNA library fragments resulting in single stranded synthesized second strands; and
subjecting the sample comprising single stranded synthesized second strands to polymerase chain reaction (PCR) amplification by contacting the sample comprising single stranded synthesized second strands with a polymerase, dNTPs, and primers complementary to the 5′ end and 3′ end library adapters under conditions to provide for second strand synthesis resulting in double stranded DNA uracil-free library fragments.

7. The method of claim 6 wherein subjecting the sample comprising single stranded synthesized second strands to PCR amplification comprises contacting the sample comprising single stranded synthesized second strands with a uracil tolerant polymerase.

8. The method of claim 1, wherein the DNA library fragments comprise:
single stranded DNA library fragments; or
double stranded DNA library fragments.

9. (canceled)

10. The method of claim 1, wherein the cytosine deaminase comprises an altered cytosine deaminase.

11-12. (canceled)

13. The method of claim 10, wherein the altered cytidine deaminase comprises an amino acid substitution mutation at a position functionally equivalent to (Tyr/Phe)130 in a wild-type APOBEC3A protein and/or an amino acid substitution mutation at a position functionally equivalent to Tyr132 in a wild-type APOBEC3A protein.

14-19. (canceled)

20. The method of claim 1, wherein the altered cytidine deaminase converts 5-methyl cytosine (5mC) to thymidine (T) by deamination at a greater rate than conversion of cytosine (C) to uracil (U) by deamination.

21. (canceled)

22. The method of claim 10, wherein the altered cytidine deaminase converts cytosine (C) to uracil (U) by deamination and 5-methyl cytosine (5mC) to thymidine (T) by deamination at a greater rate than conversion of 5-hydroxymethyl cytosine (5hmC) to 5-hydroxymethyl uracil (5hmU) by deamination.

23-24. (canceled)

25. The method of claim 10, wherein the altered cytidine deaminase is a member of the APOBEC3A subfamily and comprises a ZDD motif HXEX₂₄SW(S/T)PCX_{[2-4]}CX₆FX₈LX5R(L/I)YX_{[8-11]}LX2LX_{[10]}M (SEQ ID NO: 2), wherein the amino acid substitution mutation at the position functionally equivalent to (Tyr/Phe) 130 of the wild-type APOBEC3A protein is the Tyr (Y) amino acid of the ZDD motif.

26. (canceled)

27. The method of claim 10, wherein the altered cytidine deaminase is a member of the APOBEC3A family and comprises SEQ ID NO: 5, SEQ ID NO: 6, SEQ ID NO: 7, SEQ ID NO: 8, SEQ ID NO: 9, SEQ ID NO: 10, or SEQ ID NO: 11.

28. The method of claim 1, wherein the DNA library fragments are about 100 bp to about 300 bp in length.

29. The method of claim 1 further comprising sequencing the double stranded DNA uracil-free library fragments.

30. The method of claim 1 further comprising processing the double stranded DNA uracil-free library fragments to produce a sequencing library.

31. The method of claim 30, further comprising sequencing the sequencing library.

32-38. (canceled)
